(19) United States
(12) Patent Application Publication
(10) Pub. No.: US 2022/0226514 A1
(43) Pub. Date: Jul. 21, 2022

(54) METHODS AND COMPOSITIONS FOR DETERMINING THE BIODISTRIBUTION OF ACTIVATABLE ANTI-CD166 ANTIBODY CONJUGATES

(71) Applicant: CytomX Therapeutics, Inc., South San Francisco, CA (US)

(72) Inventors: Olga VASILJEVA, Fremont, CA (US); Danielle J. VUGTS, Amsterdam (NL); Marion CHOMET, Amsterdam (NL); Catharina Willemien Menke-van der Houven VAN OORDT, Amsterdam (NL); Guus A.M.S. VAN DONGEN, Amsterdam (NL)

(21) Appl. No.: 17/611,872

(22) PCT Filed: May 17, 2020
(86) PCT No.: PCT/US2020/033331

§ 371 Date: Nov. 16, 2021

Related U.S. Application Data
(60) Provisional application No. 62/849,714, filed on May 17, 2019.

(51) Int. Cl. A61K 51/10; C07K 16/28; A61P 35/00

(52) U.S. Cl. CPC A61K 51/1096; A61K 51/1045; A61K 51/1027; A61K 2039/505; A61P 35/00; C07K 2317/565; C07K 16/2803

(57) ABSTRACT

The present invention provides methods, compounds, and compositions useful for determining the in vivo distribution of a radionuclide after administering a radiolabeled activatable anti-CD166 antibody-bioactive agent conjugate to a subject by positron emission tomography imaging. The present invention also provides methods for identifying subjects suitable for treatment with the corresponding non-radiolabeled activatable anti-CD166-bioactive agent conjugates.

### CROSS-REFERENCE TO RELATED APPLICATIONS

This application claims the benefit of provisional application U.S. Ser. No. 62/849,714, filed May 17, 2020, pursuant 35 U.S.C. § 119(e), which is incorporated by reference in its entirety.

### FIELD OF THE INVENTION

The present invention relates to novel compounds, compositions, and related methods for detecting the biodistribution of a radiolabeled activatable anti-CD166 antibody conjugated to a bioactive agent in a subject, as well as identifying subjects suitable for treatment with the corresponding non-radiolabeled activatable anti-CD166 antibody conjugate.

### REFERENCE TO SEQUENCE LISTING

The “Sequence Listing” submitted electronically concurrently herewith pursuant to 37 C.F.R. § 1.821 in computer readable form (CFR) via EFS-Web as file name “CYTX-061-PCT_ST25” is incorporated herein by reference. The electronic copy of the Sequence Listing was created on Feb. 20, 2020, and the size on disk is 42 kilobytes.

### BACKGROUND

Antibody-based therapies have proven to be effective in the treatment of several diseases, but in some cases, toxicities due to broad target expression have limited their therapeutic effectiveness. Other limitations such as rapid clearance from the circulation following administration further hinder their effective use as a therapy. Activatable antibodies are designed to selectively activate and bind when exposed to the microenvironment of a target tissue, thus potentially reducing toxicities associated with antibody binding to widely expressed binding targets.

Methods for assessing the potential therapeutic benefit of activatable antibodies are desired.

### SUMMARY OF THE INVENTION

In one aspect, the present invention provides a method for detecting an in vivo distribution of a radiolabeled activated activatable anti-CD166 antibody-agent conjugate in a subject, the method comprising:

administering to a subject a tracer dose of a radiolabeled activatable anti-CD166 antibody-agent conjugate,
wherein the radiolabeled activatable anti-CD166 antibody-agent conjugate comprises a radionuclide coupled to an activatable anti-CD166 antibody-agent conjugate,
   wherein the activatable anti-CD166 antibody-agent conjugate comprises
   (i) an anti-CD166 antibody or an antigen binding fragment thereof (AB) that specifically binds to a mammalian (e.g., a human) CD166;
   (ii) a prodomain comprising a masking moiety (MM) and a cleavable moiety (MM), wherein the prodomain is coupled, either directly or indirectly, to the AB; and
   (iii) a bioactive agent conjugated to the AB,
wherein, when the radiolabeled activatable anti-CD166 antibody-agent conjugate is activated, a corresponding radiolabeled activated activatable anti-CD166 antibody-agent conjugate is generated that is capable of specifically binding the mammalian CD166; and

imaging the subject using positron emission tomography (PET) at a time point following administration of the tracer dose to detect the presence of the radionuclide, thereby detecting the in vivo distribution of radiolabeled activated activatable anti-CD166 antibody-agent conjugate in the subject.

In a specific embodiment, the present invention provides a method for detecting an in vivo distribution of a radiolabeled activated activatable anti-CD166 antibody-agent conjugate in a subject, the method comprising:

administering to a subject a tracer dose of a radiolabeled activatable anti-CD166 antibody-agent conjugate,
wherein the radiolabeled activatable anti-CD166 antibody-agent conjugate comprises a radionuclide coupled to an activatable anti-CD166 antibody-agent conjugate,
   wherein the activatable anti-CD166 antibody-agent conjugate comprises
   (i) an anti-CD166 antibody or an antigen binding fragment thereof (AB) that specifically binds to a human CD166;
   (ii) a prodomain comprising a masking moiety (MM) and a cleavable moiety (MM), wherein the prodomain is coupled, either directly or indirectly, to the AB; and
   (iii) a bioactive agent conjugated to the AB,
wherein, when the radiolabeled activatable anti-CD166 antibody-agent conjugate is activated, a corresponding radiolabeled activated activatable anti-CD166 antibody-agent conjugate is generated that is capable of specifically binding the human CD166; and

imaging the subject using positron emission tomography (PET) at a time point following administration of the tracer dose to detect the presence of the radionuclide, thereby detecting the in vivo distribution of radiolabeled activated activatable anti-CD166 antibody-agent conjugate in the subject.

In some embodiments, the AB comprises:

(a) a variable heavy chain complementarity determining region 1 (VH CDR1) comprising the amino acid sequence of SEQ ID NO:112;

(b) a variable heavy chain complementarity determining region 2 (VH CDR2) comprising the amino acid sequence of SEQ ID NO:113;

(c) a variable heavy chain complementarity determining region 3 (VH CDR3) comprising the amino acid sequence of SEQ ID NO:114;

(d) a variable light chain complementarity determining region 1 (VL CDR1) comprising the amino acid sequence of SEQ ID NO:115;

(e) a variable light chain complementarity determining region 2 (VL CDR2) comprising the amino acid sequence of SEQ ID NO:116; and

(f) a variable light chain complementarity determining region 3 (VL CDR3) comprising the amino acid sequence of SEQ ID NO:117.

In other embodiments, the AB comprises:

(a) a variable heavy chain complementarity determining region 1 (VH CDR1) comprising the amino acid sequence of SEQ ID NO:112;

(b) a variable heavy chain complementarity determining region 2 (VH CDR2) comprising the amino acid sequence of SEQ ID NO:113;

(c) a variable heavy chain complementarity determining region 3 (VH CDR3) comprising the amino acid sequence of SEQ ID NO:114;

(d) a variable light chain complementarity determining region 1 (VL CDR1) comprising the amino acid sequence of SEQ ID NO:124;

(e) a variable light chain complementarity determining region 2 (VL CDR2) comprising the amino acid sequence of SEQ ID NO:125; and

(f) a variable light chain complementarity determining region 3 (VL CDR3) comprising the amino acid sequence of SEQ ID NO:117.

In further embodiments, the AB comprises a heavy chain variable region comprising an amino acid sequence selected from the group consisting of SEQ ID NO:118 and SEQ ID NO:119, and a light chain variable region comprising an amino acid sequence selected from the group consisting of SEQ ID NO:120, SEQ ID NO:121, SEQ ID NO:122, and SEQ ID NO:123.

In a specific embodiment, the AB comprises a heavy chain variable region (VH) comprising the amino acid sequence of SEQ ID NO:119 and a light chain variable region (VL) comprising the amino acid sequence of SEQ ID NO:120.

In a still further embodiment, the radiolabeled activatable anti-CD166 antibody-agent conjugate comprises a light chain and a heavy chain,
wherein the light chain comprises the prodomain and a VL, and wherein the light chain comprises the amino acid sequence of SEQ ID NO:127;
wherein the heavy chain comprises the amino acid sequence of SEQ ID NO:126;

wherein the bioactive agent comprises DM4, and

wherein the radionuclide comprises ⁸⁹Zr.

In some embodiments the method further comprises administering a blocking dose to the subject, wherein the blocking dose comprises a corresponding non-radiolabeled compound selected from the group consisting of a corresponding non-radiolabeled activatable anti-CD166 antibody-agent conjugate and a corresponding non-radiolabeled activatable anti-CD166 antibody. In a specific embodiment, the blocking dose comprises a corresponding non-radiolabeled activatable anti-CD166 antibody-agent conjugate.

In another aspect, the present invention provides a method for identifying a subject suitable for treatment with an activatable anti-CD166 antibody-agent conjugate, the method comprising:

detecting the in vivo distribution of a radiolabeled activated activatable anti-CD166 antibody-agent conjugate in a subject having a tumor in accordance with any of the methods described herein; and

identifying the subject as being suitable for treatment with a corresponding non-radiolabeled activatable anti-CD166 antibody-agent conjugate if the radionuclide is detectably present within the PET image of the tumor.

In a further aspect, the present invention provides a method of treating a subject with an activatable anti-CD166 antibody-agent conjugate, the method comprising:

identifying a subject suitable for treatment with an activatable anti-CD166 antibody-agent conjugate in accordance with any of the methods described herein; and

administering to the subject a therapeutically effective dose of a corresponding non-radiolabeled activatable anti-CD166 antibody-agent conjugate.

In a still further aspect, the present invention provides a ⁸⁹Zr-labeled activatable anti-CD166 antibody-agent conjugate comprising:

⁸⁹Zr coupled via a chelation moiety to an activatable anti-CD166 antibody-agent conjugate, wherein the activatable anti-CD166 antibody-agent comprises
(i) an anti-CD166 antibody or an antigen binding fragment thereof (AB) that specifically binds to a mammalian (e.g., a human) CD166;
(ii) a prodomain comprising a masking moiety (MM) and a cleavable moiety (MM), wherein the prodomain is coupled, either directly or indirectly, to the AB; and
(iii) a bioactive agent conjugated to the AB,

wherein, when the ⁸⁹Zr-labeled activatable anti-CD166 antibody-agent conjugate is activated, a corresponding ⁸⁹Zr-labeled activated activatable anti-CD166 antibody-agent conjugate is generated that is capable of specifically binding to human CD166.

In a further aspect, the present invention provides a composition comprising the ⁸⁹Zr-labeled activatable anti-CD166 antibody-agent conjugate as described herein and a pharmaceutically acceptable carrier.

In another aspect, the present invention provides a tracer dose comprising a pharmaceutically acceptable carrier and a quantity of a ⁸⁹Zr-labeled activatable anti-CD166 antibody-agent conjugate described herein corresponding to 37 MBq.

### BRIEF DESCRIPTION OF THE FIGURES

FIG. 1A depicts the biodistribution corresponding to a ⁸⁹Zr-labeled activatable anti-CD166 antibody-agent conjugate (⁸⁹Zr-CX-2009) in H292 tumor-bearing nude mice at 72 h post-injection (p.i.) after administration of 10, 110 or 510 μg of the compound 24h post-administration of a blocking dose of 500 μg of the corresponding parental antibody (CX-090). Uptake is expressed as percentage of the injected dose per gram tissue (% ID/g) (Mean±SD, n=5 animals per group). The corresponding study is described in Example 6.

FIG. 1B depicts the biodistribution corresponding to a ⁸⁹Zr-labeled activatable anti-CD166 antibody-agent conjugate (⁸⁹Zr-CX-2009) in H292 tumor-bearing nude mice at 24, 72, and 168 h p.i. of 110 μg of the compound. Uptake is expressed as % ID/g. (Mean±SD, n=5 animals per group). The corresponding study is described in Example 6.

FIG. 2 depicts the biodistribution corresponding to ⁸⁹Zr-CX-2009 (⁸⁹Zr-labeled activatable anti-CD166 antibody-agent conjugate), ⁸⁹Zr-CX-191 (⁸⁹Zr-labeled activatable anti-CD166 antibody), ⁸⁹Zr-CX-1031 (⁸⁹Zr labeled anti-CD166 antibody-agent conjugate), and ⁸⁹Zr-CX-090 (⁸⁹Zr-labeled parental antibody) in H292 tumor-bearing nude mice, 72 h after administration of (A) 10 μg, (B) 110 μg, and (C) 510 μg, of the respective compound. Uptake is expressed as % ID/g (Mean±SD, n=5 animals per group). The corresponding study is described in Example 6.

FIG. 3 depicts the concentration of total and activated CX-2009 and CX-191 in tumor tissues from H292 xenograft mice injected with 110 μg of ⁸⁹Zr-CX-2009 and ⁸⁹Zr-CX-191 and collected at 72 p.i. Concentration is expressed as ng/ml (Mean±SD, n=5 animals per group).

FIG. 4 depicts coronal PET images of H292 tumor bearing nude mice injected with 110 jig of ⁸⁹Zr-CX-2009 and scanned at (A) 24 h, (b) 72 h, and (C) 168 h p.i. Images are decay corrected.

FIG. 5 depicts coronal PET images of H292 tumor bearing nude mice acquired 72 h p.i. of 110 μg of either (A)⁸⁹Zr-CX-2009, (B)⁸⁹Zr-CX-191, (C)⁸⁹Zr-CX-1031, or (D)⁸⁹Zr-CX-090.

### DETAILED DESCRIPTION OF THE INVENTION

The present invention provides novel compositions comprising radiolabeled activatable anti-CD166 antibody-agent conjugates and their use in assessing the biodistribution of the corresponding activated activatable anti-CD166 antibody-agent conjugate in a subject. Typically, the subject is a mammalian subject. Usually the subject is a human subject. More specifically, the present invention provides a method for detecting an in vivo distribution of a radiolabeled activated activatable anti-CD166 antibody-agent conjugate in a subject, the method comprising:

administering to a subject a tracer dose of a radiolabeled activatable anti-CD166 antibody-agent conjugate,
wherein the radiolabeled activatable anti-CD166 antibody-agent conjugate comprises a radionuclide coupled to an activatable anti-CD166 antibody-agent conjugate,
   wherein the activatable anti-CD166 antibody-agent conjugate comprises
   (i) an anti-CD166 antibody or an antigen binding fragment thereof (AB) that specifically binds to a mammalian CD166;
   (ii) a prodomain comprising a masking moiety (MM) and a cleavable moiety (MM), wherein the prodomain is coupled, either directly or indirectly, to the AB; and
   (iii) a bioactive agent conjugated to the AB,
wherein, when the radiolabeled activatable anti-CD166 antibody-agent conjugate is activated, a corresponding radiolabeled activated activatable anti-CD166 antibody-agent conjugate is generated that is capable of specifically binding the mammalian CD166; and

imaging the subject using positron emission tomography (PET) at a time point following administration of the tracer dose to detect the presence of the radionuclide, thereby detecting the in vivo distribution of radiolabeled activated activatable anti-CD166 antibody-agent conjugate in the subject. Typically, the mammalian CD166 is a human CD166.

The terms “in vivo distribution” and “biodistribution” are used interchangeably herein to refer to the location of radionuclide and associated labeled compound(s) in a mammalian subject. The terms “activatable anti-CD166 antibody”, “activatable antibody” and “AA” refer interchangeably herein to a compound that comprises: (i) an anti-CD166 antibody or an antigen binding fragment thereof (collectively referred to herein as an “AB”) that specifically binds to a human CD166; and (ii) a prodomain comprising a masking moiety (MM) and a cleavable moiety (MM), wherein the prodomain is coupled, either directly or indirectly, to the AB. As used herein, the term “prodomain” refers to a peptide which comprises a masking moiety (MM) and a cleavable moiety (CM). The terms “activatable anti-CD166 antibody-agent conjugate”, “activatable antibody conjugate”, and “AAC” are used interchangeably herein to refer to an activatable anti-CD166 antibody in which the AB is coupled to a bioactive agent. The prodomain functions to mask the AB component of the AAC until the AAC is exposed to an activation condition. Upon exposure to an activation condition, as described in more detail below, the AAC is converted to an activated AAC.

As used herein, the terms “masking moiety” and “MM”, are used interchangeably herein to refer to a peptide that, when positioned proximal to the AB, interferes with binding of the AB to a human CD166. In some embodiments, the MM interferes with binding of the AB to another mammalian CD166. An exemplary amino acid sequence for human CD166 is provided as SEQ ID NO:134. The terms “cleavable moiety” and “CM” are used interchangeably herein to refer to a peptide that is susceptible to cleavage (e.g., an enzymatic substrate, and the like), bond reduction (e.g., reduction of disulfide bond(s), and the like), or other change in physical conformation. The CM is positioned relative to the MM and AB, such that cleavage, or other change in its physical conformation, causes release of the MM from its position proximal to the AB (also referred to herein as “unmasking”).

The term “activation condition” refers to the condition that triggers unmasking of the AB, and results in generation of an “activated activatable anti-CD166 antibody-agent conjugate” or “activated AAC”. Unmasking of the AB typically results in an activated AAC having greater binding affinity for the human CD166 as compared to the corresponding AAC. The terms “peptide,” “polypeptide,” and “protein” are used interchangeably herein to refer to a polymer comprising naturally occurring or non-naturally occurring amino acid residues or amino acid analogues.

The AB may comprise one or more variable or hypervariable region of a light and/or heavy chain (VL and/or VH, respectively), variable fragment (Fv, Fab′ fragment, F(ab′)2 fragments, Fab fragment, single chain antibody (scab), single chain variable region (scFv), complementarity determining region (CDR), domain antibody (dAB), single domain heavy chain immunoglobulin of the BHH or BNAR type, single domain light chain immunoglobulins, or other polypeptide known to bind a human CD166. In some embodiments, the AB comprises an immunoglobulin comprising two Fab regions and an Fc region.

In some embodiments, an activatable antibody is multivalent, e.g., bivalent, trivalent, and so on. Thus, in some embodiments, the AA component of the AAC may comprise two or more VLs that are non-identical, and likewise, two or more VHs that are non-identical. In some embodiments, the AA component of the AAC comprises two identical VLs, each having identical sets of VL complementarity-determining regions (CDRs) and two identical VHs, each having identical sets of VH CDRs. In some of these embodiments, the AA component of the AAC comprises two identical light chains and two identical heavy chains. The assignment of amino acids to each domain is in accordance with the definitions of Kabat Sequences of Proteins of Immunological Interest (National Institutes of Health, Bethesda, Md. (1987 and 1991)); Chothia & Lesk, J. Mol. Biol. 196:901-917 (1987); Chothia, et al. Nature 342:878-883 (1989)).

ABs that are suitable for use in the practice of the present invention include those described in PCT Publication Nos. WO 2016/179285 and WO 2019/046652, both of which are incorporated herein by reference in their entireties. In a specific embodiment, the AB comprises:

(a) a variable heavy chain complementarity determining region 1 (VH CDR1) comprising the amino acid sequence of SEQ ID NO:112;

(b) a variable heavy chain complementarity determining region 2 (VH CDR2) comprising the amino acid sequence of SEQ ID NO:113;

(c) a variable heavy chain complementarity determining region 3 (VH CDR3) comprising the amino acid sequence of SEQ ID NO:114;

(d) a variable light chain complementarity determining region 1 (VL CDR1) comprising the amino acid sequence of SEQ ID NO:115;

(e) a variable light chain complementarity determining region 2 (VL CDR2) comprising the amino acid sequence of SEQ ID NO:116; and

(f) a variable light chain complementarity determining region 3 (VL CDR3) comprising the amino acid sequence of SEQ ID NO:117.

In another embodiment, the AB comprises:

(a) a variable heavy chain complementarity determining region 1 (VH CDR1) comprising the amino acid sequence of SEQ ID NO:112;

(b) a variable heavy chain complementarity determining region 2 (VH CDR2) comprising the amino acid sequence of SEQ ID NO:113;

(c) a variable heavy chain complementarity determining region 3 (VH CDR3) comprising the amino acid sequence of SEQ ID NO:114;

(d) a variable light chain complementarity determining region 1 (VL CDR1) comprising the amino acid sequence of SEQ ID NO:124;

(e) a variable light chain complementarity determining region 2 (VL CDR2) comprising the amino acid sequence of SEQ ID NO:125; and

(f) a variable light chain complementarity determining region 3 (VL CDR3) comprising the amino acid sequence of SEQ ID NO:117.

AB components suitable for use in the practice of the present invention further include those having a heavy chain variable region comprising an amino acid sequence selected from the group consisting of SEQ ID NO:118 and SEQ ID NO:119, and a light chain variable region comprising an amino acid sequence selected from the group consisting of SEQ ID NO:120, SEQ ID NO:121, SEQ ID NO:122, and SEQ ID NO:123. Typically, the AB comprises a heavy chain variable region (VH) comprising the amino acid sequence of SEQ ID NO:119 and a light chain variable region (VL) comprising the amino acid sequence of SEQ ID NO:120.

The AB component may further comprise a human immunoglobulin constant region to form a fully human IgG, such as, for example, an IgG1, an IgG2, an IgG4 or mutated constant region to form, for example, a human IgG with altered functions. Thus, the AB may further comprise a mutated Ig, such as, for example, IgG1 N297A, IgG1 N297Q, or IgG4 S228P.

In some embodiments, the radiolabeled activatable anti-CD166 antibody-agent conjugate comprises a light chain and a heavy chain,

wherein the light chain comprises the prodomain and a VL, and wherein the light chain comprises the amino acid sequence of SEQ ID NO:127; and

wherein the heavy chain comprises the amino acid sequence of SEQ ID NO:126. Often, in these embodiments, the activatable anti-CD166 antibody comprises two identical light chains and two identical heavy chains.

Masking moiety (MM) components suitable for use in the practice of the present invention include those that reduce the ability of the AB to specifically bind human CD166. As such, the dissociation constant (Kd) of the AAC toward human CD166 is usually greater than the Kd of the corresponding activated AAC to human CD166. The MM can inhibit the binding of the AAC to the human CD166 in a variety of ways. For example, the MM can bind to the AB thereby inhibiting binding of the AAC to the human CD166. The MM can allosterically or sterically inhibit binding of the AAC to human CD166. In some embodiments, the MM binds specifically to the AB. Suitable MMs may be identified using any of a variety of known techniques. For example, peptide MMs may be identified using the methods described in U.S. Patent Application Publication Nos. 2009/0062142 and 2012/0244154, and PCT Publication No. WO 2014/026136, each of which is hereby incorporated by reference in their entirety.

In some embodiments, the MM is selected such that binding of the AAC to human CD166 is reduced, relative to binding of the corresponding AB (i.e., without the prodomain) to the human CD166, by at least about 50%, or at least about 60%, or at least about 65%, or at least about 70%, or at least about 75%, or at least about 80%, or at least about 85%, or at least about 90%, or at least about 91%, or at least about 92%, or at least about 93%, or at least about 94%, or at least about 95%, or at least about 96%, or at least about 97%, or at least about 98%, or at least about 99%, and even 100%, for at least about 2 hours, or at least about 4 hours, or at least about 6 hours, or at least about 8 hours, or at least about 12 hours, or at least about 24 hours, or at least about 28 hours, or at least about 30 hours, or at least about 36 hours, or at least about 48 hours, or at least about 60 hours, or at least about 72 hours, or at least about 84 hours, or at least about 96 hours, or at least about 5 days, or at least about 10 days, or at least about 15 days, or at least about 30 days, or at least about 45 days, or at least about 60 days, or at least about 90 days, or at least about 120 days, or at least about 150 days, or at least about 180 days, or at least about 1 month, or at least about 2 months, or at least about 3 months, or at least about 4 months, or at least about 5 months, or at least about 6 months, or at least about 7 months, or at least about 8 months, or at least about 9 months, or at least about 10 months, or at least about 11 months, or at least about 12 months or more.

Typically, the MM is selected such that the Kd of the AAC towards human CD166 is at least about 2, about 3, about 4, about 5, about 10, about 25, about 50, about 100, about 250, about 500, about 1,000, about 2,500, about 5,000, about 10,000, about 100,000, about 500,000, about 1,000,000, about 5,000,000, about 10,000,000, about 50,000,000, or greater, or in the range of from about 5 to about 10, or from about 10 to about 100, or from about 10 to about 1,000, or from about 10 to about 10,000 or from about 10 to about 100,000, or from about 10 to about 1,000,000, or from about 10 to about 10 to about 10,000,000, or from about 100 to about 1,000, or from about 100 to about 10,000, or from about 100 to about 100,000, or from about 100 to about 1,000,000, or from about 100 to about 10,000,000, or from about 1,000 to about 10,000, or from about 1,000 to about 100,000, or from about 1,000 to about 1,000,000, or from about 1,000 to about 10,000,000, or from about 10,000 to about 100,000, or from about 10,000 to about 1,000,000, or from about 10,000 to about 10,000,000 or from about 100,000 to about 1,000,00, or 100,000 to about 10,000,000 times greater than the Kd of the AB (i.e., not modified with a prodomain).

Conversely, the MM is selected such that the Kd of the AB (i.e., not modified with a prodomain) towards human CD166 is at least about 2, about 3, about 4, about 5, about 10, about 25, about 50, about 100, about 250, about 500, about 1,000, about 2,500, about 5,000, about 10,000, about 100,000, about 500,000, about 1,000,000, about 5,000,000, about 10,000,000, about 50,000,000, or more times lower than the binding affinity of the corresponding AAC; or in the range of from about 5 to about 10, or from about 10 to about 100, or from about 10 to about 1,000, or from about 10 to about 10,000 or from about 10 to about 100,000, or from about 10 to about 1,000,000, or from about 10 to about 10 to about 10,000,000, or from about 100 to about 1,000, or from about 100 to about 10,000, or from about 100 to about 100,000, or from about 100 to about 1,000,000, or from about 100 to about 10,000,000, or from about 1,000 to about 10,000, or from about 1,000 to about 100,000, or from about 1,000 to about 1,000,000, or from about 1,000 to about 10,000,000, or from about 10,000 to about 100,000, or from about 10,000 to about 1,000,000, or from about 10,000 to about 10,000,000 or from about 100,000 to about 1,000,00, or 100,000 to about 10,000,000 times lower than the binding affinity of the corresponding AAC.

In some embodiments, the Kd of the MM towards the AB is greater than the Kd of the AB towards human CD166. In these embodiments, the Kd of the MM towards the AB may be at least about 5, at least about 10, at least about 25, at least about 50, at least about 100, at least about 250, at least about 500, at least about 1,000, at least about 2,500, at least about 5,000, at least about 10,000, at least about 100,000, at least about 1,000,000, or even 10,000,000 times greater than the Kd of the AB towards human CD166.

Illustrative MMs include those provided as SEQ ID NOS:84-101 and the amino acid sequence, HPL. In certain of these embodiments, the MM comprises an amino acid sequence corresponding to SEQ ID NO: 85.

Typically, the cleavable moiety (CM) component of the AACs employed herein comprise an amino acid sequence corresponding to a substrate for a protease. Usually, the protease is an extracellular protease. Suitable substrates may be readily identified using any of a variety of known techniques, including those described in U.S. Pat. Nos. 7,666,817, 8,563,269, PCT Publication No. WO 2014/026136, Boulware, et al., “Evolutionary optimization of peptide substrates for proteases that exhibit rapid hydrolysis kinetics,” Biotechnol. Bioeng. (2010) 106.3: 339-46, each of which is hereby incorporated by reference in its entirety. Exemplary substrates that are suitable for use as a cleavable moiety include, for example, those that are substrates cleavable by any one or more of the following proteases: an ADAM, an ADAM-like, or ADAMTS (such as, for example, ADAMS, ADAM9, ADAM10, ADAM12, ADAM15, ADAM17/TACE, ADAMDEC1, ADAMTS1, ADAMTS4, ADAMTS5); an aspartate protease (such as, for example, BACE, Renin, and the like); an aspartic cathepsin (such as, for example, Cathepsin D, Cathepsin E, and the like); a caspase (such as, for example, Caspase 1, Caspase 2, Caspase 3, Caspase 4, Caspase 5, Caspase 6, Caspase 7, Caspase 7, Caspase 8, Caspase 9, Caspase 10, Caspase 14, and the like); a cysteine proteinase (such as, for example, Cruzipain, Legumain, Otubain-2, and the like); a kallikrein-related peptidase (KLK) (such as, for example, KLK4, KLK5, KLK6, KLK7, KLK8, KLK10, KLK11, KLK13, KLK14, and the like); a metallo proteinase (such as, for example, Meprin, Neprilysin, prostate-specific membrane antigen (PSMA), bone morphogenetic protein 1 (BMP-1), and the like); a matrix metalloproteinase (MMP) (such as, for example, MMP1, MMP2, MMP3, MMP7, MMP8, MMP9, MMP10, MMP11, MMP12, MMP13, MMP14, MMP15, MMP16, MMP17, MMP19, MMP20, MMP23, MMP24, MMP26, MMP27, and the like); a serine protease (such as, for example, activated protein C, Cathepsin A, Cathepsin G, Chymase, a coagulation factor protease (such as, for example, FVIIa, FIXa, FXa, FXIa, FXIIa, and the like)); elastase, Granzyme B, Guanidinobenzoatase, HtrA1, Human Neutrophil Elastase, Lactoferrin, Marapsin, NS3/4A, PACE4, Plasmin, prostate-specific antigen (PSA), tissue plasminogen activator (tPA), Thrombin, Tryptase, urokinase (uPA), a Type II transmembrane Serine Protease (TTSP) (such as, for example, DESC1, DPP-4, FAP, Hepsin, Matriptase-2, MT-SP1/Matriptase, TMPRSS2, TMPRSS3, TMPRSS4, and the like), and the like. Exemplary CMs that are suitable for use in practice of the present invention include those described in, for example, WO 2010/081173, WO 2015/048329, WO 2015/116933, and WO 2016/118629, each of which is incorporated herein by reference in its entirety. Illustrative CMs are provided herein as SEQ ID NOs: 1-67. Thus, in some embodiments, the radiolabeled AAC comprises (i.e., has a prodomain comprising) a CM that comprises an amino acid sequence selected from the group consisting of SEQ ID NOs:1-67. In some embodiments, the CM comprises an amino acid sequence corresponding to SEQ ID NO:25.

The AA component of the AACs employed herein may comprise the AB and prodomain components, CM and MM, in a variety of linear or cyclic configurations (via, for example, a cysteine-cysteine disulfide bond), and may further comprise one or more optional linker moieties through which any two or more of the AB, CM, and/or MM moieties may be bound indirectly to each other. Linkers suitable for use in the AACs employed in the practice of the invention may be any of a variety of lengths. Suitable linkers include those having a length in the range of from about 1 to about 20 amino acids, or from about 1 to about 19 amino acids, or from about 1 to about 18 amino acids, or from about 1 to about 17 amino acids, or from about 1 to about 16 amino acids, or from about 1 to about 15 amino acids, or from about 2 to about 15 amino acids, or from about 3 to about 15 amino acids, or from about 3 to about 14 amino acids, or from about 3 to about 13 amino acids, or from about 3 to about 12 amino acids. In some embodiments, the AA component of the AAC comprises one or more linkers comprising 1, 2, 3, 4, 5, 6, 7, 8, 9, 10, 11, 12, 13, 14, 15, 16, 17, 18, 19, or 20 amino acids. Typically, the linker is a flexible linker. As used herein, the term “range” is intended to be inclusive of the endpoints which define the limits of the range.

Exemplary flexible linkers include glycine homopolymers (G)ₙ, (wherein n is an integer that is at least 1; in some embodiments, n is an integer in the range of from about 1 to about 30, or an integer in the range of from about 1 to about 25, or an integer in the range of from about 1 to about 20, or an integer in the range of from about 1 to about 20, or an integer in the range of from about 1 to about 15, or an integer in the range of from about 1 to about 10), glycine-serine polymers, including, for example, (GS). (wherein n is an integer that is at least 1), (GSGGS)ₙ (SEQ ID NO:68) (wherein n is an integer that is at least 1; in some embodiments, n is an integer in the range of from about 1 to about 30, or an integer in the range of from about 1 to about 25, or an integer in the range of from about 1 to about 20, or an integer in the range of from about 1 to about 20, or an integer in the range of from about 1 to about 15, or an integer in the range of from about 1 to about 10), (GGGS)ₙ (SEQ ID NO:69) (wherein n is an integer that is at least 1; in some embodiments, n is an integer in the range of from about 1 to about 30, or an integer in the range of from about 1 to about 25, or an integer in the range of from about 1 to about 20, or an integer in the range of from about 1 to about 20, or an integer in the range of from about 1 to about 15, or an integer in the range of from about 1 to about 10), GGSG (SEQ ID NO:70), GGSGG (SEQ ID NO:71), GSGSG (SEQ ID NO:72), GSGGG (SEQ ID NO:73), GGGSG (SEQ ID NO:74), GSSSG (SEQ ID NO:75), GSSGGSGGSGGSG (SEQ ID NO:76), GSSGGSGGSGG (SEQ ID NO:77), GSSGGSGGSGGS (SEQ ID NO:78), GSSGGSGGSGGSGGGS (SEQ ID NO:79), GSSGGSGGSG (SEQ ID NO:80), GSSGGSGGSGS (SEQ ID NO:81), GGGS (SEQ ID NO:69), GSSGT (SEQ ID NO:82), GSSG (SEQ ID NO:83), GGGSSGGSGGSGG (SEQ ID NO:128), GGS, and the like, and additionally, a glycine-alanine polymer, an alanine-serine polymer, and other flexible linkers known in the art. In some embodiments, the prodomain is linked indirectly to the AB via a linker comprising an amino acid sequence selected from the group consisting of any one of SEQ ID NOs:69-83, 128, SGS, GS, S, GQG, QG, G, SGQ, GQ, and Q. In certain embodiments, the MM and CM of the prodomain are coupled indirectly to each other via a linker having an amino acid sequence selected from the group consisting of any one of SEQ ID NOs:69-83, 128, SGS, GS, S, GQG, QG, G, SGQ, GQ, and Q. In other embodiments, the prodomain is linked indirectly to the AB via a linker comprising an amino acid sequence selected from the group consisting of any one of SEQ ID NOs:68-83.

Illustrative structural arrangements of MM, CM, AB, and linker (L) components in the AA portion of the AAC include, for example, in either N- to C-terminal direction or C- to N-terminal direction:
(MM)-(CM)-(AB)
(AB)-(CM)-(MM)
(MM)-L₁-(CM)-(AB)
(MM)-L₁-(CM)-L₂-(AB)
cyclo [L₁-(MM)-L₂-(CM)-L₃-(AB)]

wherein each of L₁, L₂, and L₃ is a linker peptide that may be identical or different.

The AA component of the AAC may also include a spacer located, for example, at the amino terminus of the prodomain. In some embodiments, the spacer is joined directly to the MM of the prodomain. In some embodiments, the spacer is joined directly to the MM of the prodomain in the structural arrangement from N-terminus to C-terminus of spacer-MM-CM-AB. An example of a spacer joined directly to the N-terminus of MM of the activatable antibody is selected from the group consisting of QGQSGQ (SEQ ID NO:102), QGQSGQG (SEQ ID NO:103), QGQSG (SEQ ID NO:104), QGQS (SEQ ID NO:105), GQSGQG (SEQ ID NO:106), QSGQG (SEQ ID NO:107), SGQG (SEQ ID NO:108), GQSGQG (SEQ ID NO:109), QSGQG (SEQ ID NO:110), SGQG (SEQ ID NO:111), QGQSGS (SEQ ID NO:129), GQSGS (SEQ ID NO:130), QSGS (SEQ ID NO:131), GQSGQ (SEQ ID NO:132), QSGQ (SEQ ID NO:133), SGS, GS, S, GQG, QG, G, SGQ, GQ, and Q. Often the spacer has the amino acid sequence of SEQ ID NO:103.

Typically, the prodomain is linked, either directly or indirectly, to the AB via the CM of the prodomain. The CM may be designed to be cleaved by upregulated proteolytic activity (i.e., the activation condition) in tissue, such as those present in many cancers. Thus, AACs may be designed so they are predominantly activated at a target treatment site where proteolytic activity and the desired mammalian (e.g., human) CD166 are co-localized.

As used herein, the term “bioactive agent” refers to an agent that, when administered to a subject, has a biological effect on the subject. In some embodiments, the biological effect is the alleviation or delay in the progression of a cancer. Suitable bioactive agents include those selected from the group consisting of a cytotoxic agent (such as, for example, an auristatin (e.g., auristatin E, monomethyl auristatin D (MMAD), monomethyl auristatin E (MMAE), desmethyl auristatin E (DMAE), auristatin F, monomethyl auristatin F (MMAF), desmethyl auristatin F (DMAF), auristatin tyramine, auristatin quinoline, and the like, as well as other auristatin derivatives, such as, for example, amide derivatives, and the like), a dolastatin (such as, for example, dolastin 16 DmJ, dolastin 16 Dpv, and the like, as well as other dolastin derivatives), a maytansinoid (such as, for example, DM1, DM4, and the like, as well as other maytansinoid derivatives), a duocarmycin (including any derivatives thereof), an amanitin (such as, for example, alpha-amanitin, and the like), an anthracycline, doxorubicin, caunorubicin, a bryostatin, a camptothecin (such as, for example, 7-substituted camptothecin, 10,11-difluoromethylenedioxycamptothecin, and the like, as well as other camptothecin derivatives), a combretastatin, a debromoaplysiatoxin, kahalalide-F, discodermolide, an ecteinascidins, a turbostatin, a phenstatin (such as, for example, hydroxyphenstatin, and the like), a spongistatin (such as, for example, spongistatin 5, spongistatin 7, and the like), a halistatin (such as, for example, halistatin 1, halistatin 2, halistatin 3, and the like), a bryostatin, a halocomstatin, a pyrrolobenzimidazole, cibrostatin6, doxaliform, an anthracycline, a cemadotin (such as, for example, CemCH2-SH, and the like), a Pseudomonas toxin A (such as, for example, Pseudomonas toxin A (PE38) variant, Pseudomonas toxin A (ZZ-PE38) variant, and the like), a superstolide A (such as, for example, ZJ-101, and the like), a saponin (such as, for example, OSW-1, and the like), an O6-benzylguanine, a topoiosomerase inhibitor, a hemiasterlin, a cephalotaxine, a hemoharringtonine, a pyrrolobenzodiazepene, a calicheamicin, a podophyllotoxin, a taxane, and a vinca alkaloid), an antiviral agent (such as, for example, acyclovir, Vira A, Symmetrel, and the like), an antifungal agent (such as, for example, nystatin, and the like), an anti-neoplastic agent (such as, for example, adriamycin, cerubidine, bleomycin, alkeran, velban, oncovin, fluorouracil, methotrexate, thiotepa, bisantrene, novantrone, thioguanine, procarabizine, cytarabine, and the like), a heavy metal (such as, for example, barium, gold, platinum, and the like), an anti-bacterial agent (such as, for example, an aminoglycoside, streptomycin, neomycin, kanamycin, amikacin, gentamicin, tobramycin, streptomycin B, spectinomycin, ampicillin, sulfanilamide, polymyxin, chloramphenicol, and the like), an antimycoplasmal agent (such as, for example, tylosine, spectinomycin, and the like), and the like.

Often the bioactive agent is a cytotoxic agent. In some embodiments, the bioactive agent is a maytansinoid. In certain embodiments, the bioactive agent is DM4.

The bioactive agent is typically conjugated to the AB using a conjugation linker and methods that are known in the art. Conjugation linkers that are suitable for use in the AACs employed herein include those described in PCT Publication Nos. WO 2016/179285 and WO 2019/046652, and Ramakrishnan, S. et al., Cancer Res. 44:201-208 (1984), U.S. Pat. No. 5,030,719, each of which is incorporated herein by reference in their entireties. Exemplary conjugation linkers that are suitable for conjugating the bioactive agent to the AA include: (i) EDC (1-ethyl-3-(3-dimethylamino-propyl) carbodiimide hydrochloride; (ii) SMPT (4-succinimidyloxycarbonyl-alpha-methyl-alpha-(2-pridyl-dithio)-toluene (Pierce Chem. Co., Cat. (21558G); (iii) SPDP (succinimidyl-6 [3-(2-pyridyldithio) propionamido]hexanoate (Pierce Chem. Co., Cat #21651G); (iv) Sulfo-LC-SPDP (sulfosuccinimidyl 6 [3-(2-pyridyldithio)-propianamide] hexanoate (Pierce Chem. Co. Cat. #2165-G); and (v) sulfo-NHS (N-hydroxysulfo-succinimide: Pierce Chem. Co., Cat. #24510) conjugated to EDC. Additional linkers include, but are not limited to, SMCC ((succinimidyl 4-(N-maleimidomethyl)cyclohexane-1-carboxylate), sulfo-SMCC (sulfosuccinimidyl 4-(N-maleimidomethyl)cyclohexane-1-carboxylate), SPDB (N-succinimidyl-4-(2-pyridyldithio) butanoate), or sulfo-SPDB (N-succinimidyl-4-(2-pyridyldithio)-2-sulfo butanoate). Often, the conjugation linker is SPDB. In certain embodiments, the AAC comprises the bioactive agent, DM4, conjugated to the AA via the conjugation linker SPDB.

In some embodiments, the AA is conjugated to one or more equivalents of a biological agent. In some embodiments, the AA is conjugated to one equivalent of the bioactive agent. In some embodiments, the AA is conjugated to two, three, four, five, six, seven, eight, nine, ten, or greater than ten equivalents of the bioactive agent. In some embodiments, the AA is part of a mixture of AAs having a homogeneous number of equivalents of conjugated bioactive agents. In some embodiments, the AA is part of a mixture of AAs having a heterogeneous number of equivalents of conjugated bioactive agents. In some embodiments, the mixture of AAs is such that the average number of bioactive agents conjugated to each AA is between zero to one, between one to two, between two and three, between three and four, between four and five, between five and six, between six and seven, between seven and eight, between eight and nine, between nine and ten, and ten and greater. In some embodiments, the mixture of AAs is such that the average number of bioactive agents conjugated to each AA is one, two, three, four, five, six, seven, eight, nine, ten, or greater. In some embodiments, there is a mixture of AAs such that the average number of bioactive agents conjugated to each AA is between three and four. In some embodiments, there is a mixture of AAs such that such that the average number of agents conjugated to each AA is between 3.4 and 3.8. In some embodiments, there is a mixture of AAs such that such that the average number of agents conjugated to each AA is between 3.4 and 3.6. In some embodiments, the AA comprises one or more site-specific amino acid sequence modifications such that the number of lysine and/or cysteine residues is increased or decreased with respect to the original amino acid sequence of the activatable antibody, thus in some embodiments correspondingly increasing or decreasing the number of bioactive agents that can be conjugated to the activatable antibody, or in some embodiments limiting the conjugation of the bioactive agents to the AA in a site-specific manner. In some embodiments, the modified AA is modified with one or more non-natural amino acids in a site-specific manner, thus in some embodiments limiting the conjugation of the bioactive agents to only the sites of the non-natural amino acids.

Radionuclides that are suitable for use in the radiolabeled AACs employed herein include any that are suitable for use in positron emission tomography. These include, for example, ¹¹¹In (half-life 67.3 hours), ¹³¹I (half-life 192.5 hours), ¹²³I (half-life 13.2 hours), ^{99m}Tc (half-life 6.0 hours), ¹⁷⁷Lu (half-life 159.5 hours), ⁸⁹Zr (half-life 78.4 hours), 124I (half-life 100.2 hours), ⁶⁴Cu (half-life 12.7 hours), ⁸⁶Y (half-life 14.7 hours), ⁷⁰Br (half-life 16.1 hours), ¹⁸F (half-life 1.83 hours), 68Ga (half-life 1.13 hours), and the like. Often, the radionuclide is ⁸⁹Zr.

The radiolabeled AAC is often prepared by reacting the corresponding AA with a labeling moiety. As used herein, the term “labeling moiety” is a moiety that is capable of forming bonds with both the radionuclide and the AA portion of the AAC. Typically, conjugation of the labeling moiety to the AA is via a covalent bond. In an exemplary embodiment, the labeling moiety comprises a chelation moiety. The term “chelation moiety” refers to a moiety that is capable of forming one or more bonds with the radionuclide. In these embodiments, the radiolabeled AAC further comprises a chelation moiety to which the radionuclide is chelated. When a chelation moiety is employed, it is conjugated to an amino acid residue in the activatable antibody. The chelation moiety may comprise a further substituent to facilitate and direct conjugation to the AA portion of the AAC.

Exemplary AACs that comprise chelation moieties include those which result from reaction of the AAC with chelation agents such as, for example, diethylenetriaminepentaacetic acid (DTPA), ethylenediaminetetraacetic acid (EDTA), 1,4,7,10-tetraacetic acid (DOTA), desferrioxamine (DFO), and the like. Thus, the structure of the chelation moiety corresponds to the structure of the structure of the chelation agent with the exception of the portion of the chelation agent that is conjugated to the amino acid residue of the AA portion of the AAC. Thus, in some embodiments, the chelation moiety may comprise a structure corresponding to a chelation agent selected from the group consisting of diethylenetraminepentaacetic acid, ethylenediaminetetraacetic acid, 1,4,7,10-tetraacetic acid, and desferrioxamine. Often, the radiolabeled AAC comprises a chelation moiety comprising a structure corresponding to desferrioxamine.

Known methods for preparing radiolabeled antibodies using chelation agents are suitable for preparing the radiolabeled AACs employed herein. These methods are described in, for example, Chan, et al., Pharmaceuticals (2012) 5:79-91, van de Watering, et al., BioMed Research International Vol. 2014, Article ID 203601 (2014), Zhang, et al., Curr. Radiopharm. (2011) 4(2):131-139, and LeBeau, et al., Cancer Res. (2015) 75(7):1225-1235, Verel, et al., J. Nucl. Med. (2003) 44:1271-1281, Vosjan, et al., Eur. J. Nucl. Med. Mol. Imaging (2011) 38:753-763, Vosjan, et al., “Conjugation and Radiolabeling of Monoclonal Antibodies with Zirconium-89 for PET Imaging Using the Bifunctional Chelate p-Isothiocyanatobenzyl-Desferrioxamine, Nat. Protoc. (2010) 5(4), 739-743, each of which is incorporated herein by reference in their entireties.

The dose of a radiolabeled AAC (i.e., the “tracer” dose) is often administered in the form of a composition comprising a radiolabeled AAC and one or more of a suitable carrier, an excipient, and/or other agent(s) that are incorporated into pharmaceutical formulations to provide improved transfer, delivery, tolerance, stability, and the like. In some embodiments, the carrier is a physiological saline solution (i.e., 0.9% NaCl), a saccharide solution (e.g., dextrose, and the like), an alcohol (e.g., ethanol), a polyol (e.g., a polyalcohol, such as, for example, mannitol, sorbitol, and the like), a glycol, such as ethylene glycol, propylene glycol, polyethylene glycol (PEG), a coating agent, an isotonic agent, such as mannitol or sorbitol, an organic ester, such as ethyoleate, an absorption-delaying agent, such as aluminum monostearate and gelatins and the like, as well as mixtures of any two or more thereof. The composition can be in the form of a stable, aqueous solution. The aqueous solution may comprise an isotonic vehicle such as sodium chloride, Ringer's injection solution, dextrose, lactated Ringer's injection solution, or equivalent delivery vehicle (e.g., sodium chloride/dextrose injection solution). The composition may comprise aqueous and non-aqueous, isotonic sterile injection solutions, which can include solvents, co-solvents, antioxidants, reducing agents, chelating agents, buffers, bacteriostats, antimicrobial preservatives and solutes that render the composition isotonic with the blood of the intended recipient (e.g., PBS and/or saline solutions, such as 0.1 M NaCl) and aqueous and non-aqueous sterile suspensions that can include suspending agents, solubilizers, thickening agents, emulsifying agents, stabilizer, preservatives, and the like. Suitable agents can be found in Remington's Pharmaceutical Science (15th ed. Mack Publishing Company, Easton, Pa. (1975)), which is incorporated herein by reference in its entirety.

In some embodiments, the tracer dose is about 37 MBq. The tracer dose is typically administered in the form of a composition comprising the radiolabeled AAC and a pharmaceutically acceptable carrier, such as any of those described hereinabove. The carrier in the composition of the tracer dose (i.e., “tracer dose composition”) is typically a liquid phase carrier. Typically, the mammalian subject is a human or non-human mammal suspected of having a disease or disorder. Often, the subject is a human. Usually the suspected disease or disorder is a cancer, as described in more detail hereinbelow. In some embodiments, the subject has a solid tumor.

In some embodiments, the method further comprises administering a blocking dose to the subject, wherein the blocking dose comprises a corresponding non-radiolabeled (i.e., “cold”) compound selected from the group consisting of a corresponding non-radiolabeled activatable anti-CD166 antibody-agent conjugate and a corresponding non-radiolabeled activatable anti-CD166 antibody. Usually, the blocking dose comprises a corresponding non-radiolabeled activatable anti-CD166 antibody-agent conjugate. Typically, the administering of the blocking dose precedes the administering of the tracer dose to pre-block non-specific antigen sinks. In some embodiments, the blocking dose comprises from about 0.25 mg/kg to about 10 mg/kg, or from about 0.25 mg/kg to about 6 mg/kg, or from about 6 mg/kg to about 10 mg/kg of the corresponding non-radiolabeled activatable anti-CD166 antibody-agent conjugate. As used herein, the term “corresponding non-radiolabeled activatable anti-CD166 antibody-agent conjugate” refers to a compound have the same AAC structure as the referenced radiolabeled AAC, but without the radiolabel.

After administering the tracer dose, subjects are subjected to positron emission tomography (PET) scanning at one or more time-points. Typically, the imaging step is carried out in the period of from about 1 day to about 10 days post tracer dose administration. In some embodiments, the treated subject is subjected to PET scanning at a time point in the period of from about 2 days to about 10 days post tracer dose administration, or in the period of from about 2 days to about 9 days post tracer dose administration, or in the period of from about 2 days to about 8 days post tracer dose administration, or in the period of from about 2 days to about 7 days post tracer dose administration, or in the period of from about 3 days to about 10 days post tracer dose administration, or in the period of from about 3 days to about 9 days post tracer dose administration, or in the period of from about 3 days to about 8 days post tracer dose administration. In certain embodiments, the treated subject is subjected to PET scanning at day 2, and/or day 4, and/or day 7 post tracer dose administration. In other embodiments, the treated subject is subjected to PET scanning at day 1, and/or day 3, and/or day 6 post tracer dose administration.

Typically, the resulting PET scan covers an area that includes one or more organs or tissue corresponding to the heart, blood, lung, liver, kidney, pancreas, stomach, ilium, colon, muscle, bone, skin, brain, thymus, brown adipose tissue (BAT), spleen, and/or tumor. Usually the PET scan covers an area that includes all or a portion of a tumor. In some embodiments, the PET scan covers an area that includes all or a portion of a tumor and all or a portion of at least one other organ or tissue type. In some embodiments, the PET scan covers the whole body of the subject.

Detection of radionuclide in the PET scan indicates the presence of AAC and the location and thus the in vivo biodistribution of activated AAC in the mammalian subject. Detection of activated AAC indicates not only that the administered AAC was activated, e.g., by proteases in the target microenvironment, but that the mammalian (e.g., human) CD166 was also present. Thus, the method may be further used to identify subjects more likely to benefit from treatment with a particular AAC. For example, if the biodistribution indicates the presence of radiolabled activated AAC in a tumor, the subject may be more likely to benefit from the administration of the AAC for the treatment of the tumor and associated cancer. Therefore, the present invention further provides a method for identifying a subject suitable for treatment with an activatable anti-CD166 antibody-agent conjugate, the method comprising:

detecting the in vivo distribution of a radiolabeled activated activatable anti-CD166 antibody-agent conjugate in a subject having a tumor in accordance with any of the methods described herein; and

identifying the subject as being suitable for treatment with a corresponding non-radiolabeled activatable anti-CD166 antibody-agent conjugate if the radionuclide is detectably present within the PET image of the tumor. In some embodiments it may be desired to further obtain a tumor tissue sample from the subject.

In a further embodiment, the present invention provides a method of treating a subject with an activatable anti-CD166 antibody-agent conjugate, the method comprising:

identifying a subject suitable for treatment with an activatable anti-CD166 antibody-agent conjugate as described above, and

administering to the subject a therapeutically effective dose of a corresponding non-radiolabeled activatable anti-CD166 antibody-agent conjugate.

As used herein, the term “therapeutically effective dose” refers to the quantity of non-radiolabeled activatable anti-CD166 antibody-agent conjugate effective in alleviating a symptom of a disease or disorder when administered either once, or in a series over a period of time. Typically, the disease or disorder is a cancer. In some embodiments, the therapeutically effective dose is from about 0.25 mg/kg to about 10 mg/kg, or from about 0.25 mg/kg to about 6 mg/kg, or from about 6 mg/kg to about 10 mg/kg of the corresponding non-radiolabeled activatable anti-CD166 antibody-agent conjugate. Suitable therapeutically effective doses of activatable anti-CD166 antibody-agent conjugates are described in WO 2019/046652, which is incorporated herein by reference in its entirety.

In one embodiment, the mammalian subject has been previously diagnosed with a disease or disorder, such as cancer. Exemplary types of cancer, include, for example, an advanced, unresectable solid tumor or lymphoma (e.g., a PDL1-responsive tumor type); a carcinoma such as, for example, carcinoma squamous cell carcinoma, an anal squamous cell carcinoma, gastric carcinoma, bowel carcinoma (such as, for example, small bowel carcinoma or small bowel adenocarcinoma), hepatocellular carcinoma, or a basal cell carcinoma; bladder cancer; bone cancer; breast cancer, such as, for example, triple negative breast cancer (TNBC) or estrogen receptor positive breast cancer; a carcinoid; castration-resistant prostate cancer (CRPC), cervical carcinoma, colon cancer (such as, for example, a colon adenocarcinoma); cutaneous squamous cell carcinoma, colorectal cancer (CRC), endometrial cancer, esophageal cancer, gastroesophageal junction cancer, glioblastoma/mixed glioma, glioma, head and neck cancer, hematologic malignancy, such as, for example, a lymphoma (such as, for example, a B-cell lymphoma, a T-cell lymphoma, Hodgkin's lymphoma, an EBV lymphoma, or a primary mediastinal B-cell lymphoma) or a leukemia; liver cancer, lung cancer (such as, for example, non-small cell lung cancer (NSCLC) (such as, for example, non-squamous NSCLC or squamous NSCLC) or small cell lung cancer); melanoma, Merkel cell carcinoma, multiple myeloma, nasopharyngeal cancer, osteosarcoma, ovarian cancer, pancreatic cancer, peritoneal carcinoma, undifferentiated pleomorphic sarcoma, prostate cancer (such as, for example, small cell neuroendocrine prostate cancer); rectal carcinoma, renal cancer (such as, for example, a renal cell carcinoma or a renal sarcoma); sarcoma, salivary gland carcinoma, squamous cell carcinoma, stomach cancer, testicular cancer, thymic carcinoma, thymic epithelial tumor, thymoma, thyroid cancer, urogenital cancer, urothelial cancer, uterine carcinoma, uterine sarcoma, and the like. In some embodiments, the cancer is a High Tumor Mutational Burden (hTMB) cancer.

In another aspect, the present invention provides a ⁸⁹Zr-labeled activatable anti-CD166 antibody-agent conjugate comprising:

⁸⁹Zr coupled via a chelation moiety to an activatable anti-CD166 antibody-agent conjugate, wherein the activatable anti-CD166 antibody-agent comprises
(i) an anti-CD166 antibody or an antigen binding fragment thereof (AB) that specifically binds to a mammalian (e.g., human) CD166;
(ii) a prodomain comprising a masking moiety (MM) and a cleavable moiety (MM), wherein the prodomain is coupled, either directly or indirectly, to the AB; and
(iii) a bioactive agent conjugated to the AB,

wherein, when the ⁸⁹Zr-labeled activatable anti-CD166 antibody-agent conjugate is activated, a corresponding ⁸⁹Zr-labeled activated activatable anti-CD166 antibody-agent conjugate is generated that is capable of specifically binding to human CD166. As described hereinabove, such compounds are useful as tracers in connection with PET imaging a tumor in a mammalian subject.

In certain specific embodiments, the ⁸⁹Zr-labeled activatable anti-CD166 antibody-agent conjugate comprises a chelation moiety having a structure corresponding to desferrioxamine. In some embodiments, the ⁸⁹Zr-labeled activatable anti-CD166 antibody-agent has an AB that comprises:
(a) a variable heavy chain complementarity determining region 1 (VH CDR1) comprising the amino acid sequence of SEQ ID NO:112;
(b) a variable heavy chain complementarity determining region 2 (VH CDR2) comprising the amino acid sequence of SEQ ID NO:113;
(c) a variable heavy chain complementarity determining region 3 (VH CDR3) comprising the amino acid sequence of SEQ ID NO:114;
(d) a variable light chain complementarity determining region 1 (VL CDR1) comprising the amino acid sequence of SEQ ID NO:115;
(e) a variable light chain complementarity determining region 2 (VL CDR2) comprising the amino acid sequence of SEQ ID NO:116;
(f) a variable light chain complementarity determining region 3 (VL CDR3) comprising the amino acid sequence of SEQ ID NO:117.

In other embodiments, the ⁸⁹Zr-labeled activatable anti-CD166 antibody-agent has an AB that comprises:
(a) a variable heavy chain complementarity determining region 1 (VH CDR1) comprising the amino acid sequence of SEQ ID NO:112;
(b) a variable heavy chain complementarity determining region 2 (VH CDR2) comprising the amino acid sequence of SEQ ID NO:113;
(c) a variable heavy chain complementarity determining region 3 (VH CDR3) comprising the amino acid sequence of SEQ ID NO:114;
(d) a variable light chain complementarity determining region 1 (VL CDR1) comprising the amino acid sequence of SEQ ID NO:124;
(e) a variable light chain complementarity determining region 2 (VL CDR2) comprising the amino acid sequence of SEQ ID NO:125;
(f) a variable light chain complementarity determining region 3 (VL CDR3) comprising the amino acid sequence of SEQ ID NO:117.

In some embodiments, the ⁸⁹Zr-labeled activatable anti-CD166 antibody-agent conjugate has an AB that comprises a heavy chain variable region comprising an amino acid sequence selected from the group consisting of SEQ ID NO:118 and SEQ ID NO:119, and a light chain variable region comprising an amino acid sequence selected from the group consisting of SEQ ID NO:120, SEQ ID NO:121, SEQ ID NO:122, and SEQ ID NO:123. Often, the ⁸⁹Zr-labeled activatable anti-CD166 antibody-agent conjugate has an AB that comprises a heavy chain variable region comprising the amino acid sequence of SEQ ID NO:119 and a light chain variable region comprising the amino acid sequence of SEQ ID NO:120.

The ⁸⁹Zr-labeled activatable anti-CD166 antibody-agent conjugates of the present invention may have a prodomain that comprises an MM which in turn comprises an amino acid sequence selected from the group consisting of any one of SEQ ID NOs:84-99 and HPL. In some embodiments, the prodomain comprises a CM that comprises an amino sequence selected from the group consisting of any one of SEQ ID NOs:1-67. The prodomain of the ⁸⁹Zr-labeled activatable anti-CD166 antibody-agent conjugate may further comprise a spacer comprising an amino acid sequence selected from the group consisting of any one of SEQ ID NOs:102-111 and 129-133.

In a specific embodiment, the ⁸⁹Zr-labeled activatable anti-CD166 antibody-agent conjugate of the present invention has an activatable anti-CD166 antibody-agent conjugate component that comprises a light chain and a heavy chain,

wherein the light chain comprises the prodomain and a VL, and wherein the light chain comprises the amino acid sequence of SEQ ID NO:127; and

wherein the heavy chain comprises the amino acid sequence of SEQ ID NO:126. In some embodiments, the bioactive agent comprises DM4.

In certain embodiments, the ⁸⁹Zr is coupled to the activatable anti-CD166 antibody-agent conjugate via a chelation moiety having a structure corresponding to desferrioxamine. Often, the ⁸⁹Zr-labeled activatable anti-CD166 antibody-agent conjugate has an AA that comprises two identical light chains and two identical heavy chains.

In a further embodiment, the present invention provides a composition comprising any of the ⁸⁹Zr-labeled activatable anti-CD166 antibody-agent conjugates described herein and a pharmaceutically acceptable carrier. Suitable carriers that may be employed in the practice of the present invention may be found in Remington's Pharmaceutical Science (15th ed. Mack Publishing Company, Easton, Pa. (1975)), which is incorporated herein by reference in its entirety. The compositions may further comprise a corresponding non-radiolabeled AAC.

In one embodiment, the composition comprises the radiolabeled AAC and a solid phase carrier. In these embodiments, the composition is typically in lyophilized form. Prior to administering the radiolabeled AAC to the mammalian subject, the composition is reconstituted to a solution form by addition of a liquid to form the tracer dose composition, where the tracer dose composition comprises the radiolabeled AAC at the desired quantity in the tracer dose. Typically, the liquid is physiological saline (0.9% NaCl). The term “tracer dose composition” refers to the composition of the tracer dose that is administered to the mammalian subject. In other embodiments, the composition comprises the radiolabeled AAC and a liquid phase carrier. This composition may be the tracer dose composition, or it may be a composition that is diluted by addition of a liquid, e.g., physiological saline (0.9% NaCl), to a tracer dose composition comprising the radiolabeled AAC at the desired quantity in the tracer dose.

The following examples further illustrate the invention but should not be construed as limiting its scope in any way.

### EXAMPLES

### Example 1

### Activatable Anti-CD166 Antibody-Agent Conjugate and Related Compounds

An activatable anti-CD166 antibody-agent conjugate (CX-2009) having a heavy chain of SEQ ID NO:126 and a light chain of SEQ ID NO:127 conjugated to DM4 via an N-succinimidyl-4-(2-pyridyldithio) butanoate (SPDB) linker was prepared in accordance with the description provided in PCT Publication Nos. WO 2016/179285 and WO 2019/046652, both of which are incorporated herein by reference in their entireties. The activatable anti-CD166 antibody-agent conjugate has an average of 3.5 DM4 molecules coupled per activatable anti-CD166 antibody agent conjugate molecule. Three additional compounds were prepared: the parental MAb component of CX-2009 (“CX-090”); the parental antibody component of CX-2009 conjugated with an average of with 3.7 DM4 molecules per antibody (“CX-1031”); and the activatable anti-CD166 antibody component of CX-2009 without DM4 (“CX-191”).

The CD166 binding properties of these molecules was characterized by an ELISA-based assay. 96-well plates (Nunc Maxisorp, Thermo Fisher) were coated with 200 ng/well of recombinant CD166 protein in 0.05 M carbonate buffer. Plates were washed 3×300 μl in TBS, 0.1% Tween (wash buffer) then blocked with TBS+0.5% casein (block) for 1 hr at room temperature. Plates were washed 3× and incubated in 80 μl of indicated concentrations of CX-090, CX-191, CX-1031 or CX-2009 for 1 hr at room temperature. Plates were washed and incubated with 80 μl of detection antibody (AffiniPure Anti-human IgG, Jackson ImmunoResearch cat #109-088) at 1 to 10,000 dilution in block for 30-45 min. at room temperature. Detection was performed by the addition of 3,3′,5,5′-tetramethylbenzidine substrate (1-Step Ultra-TMB, Pierce) followed by an equal volume of 1M hydrochloric acid. Absorbance at 450 nm was then measured and reported as optical density (OD 450 nm). Data were graphed in Prism Graphpad, and apparent equilibrium binding constants (Kapp) were determined using non-linear regression four parameter logistic (4-PL) analysis.

### Example 2

### Preparation of ⁸⁹Zr-Labeled Activatable Anti-CD166-Agent Conjugate and Derivatives

### A. ⁸⁹Zr-CX-2009

Five mg of CX-2009 (5.3 mg/ml) were diluted to a 5 mg/mL solution with 0.9% NaCl, adjusted to pH=8.9-9.1 by addition of a ±130 μL 0.1 M Na₂CO₃, and reacted with 5 equivalents of the bifunctional chelator DFO-NCS in DMSO (5 mM, 32 μL) at 37° C. for 30 min, essentially as described by Vosjan, et al., “Conjugation and Radiolabeling of Monoclonal Antibodies with Zirconium-89 for PET Imaging Using the Bifunctional Chelate p-Isothiocyanatobenzyl-Desferrioxamine, Nat. Protoc. (2010) 5(4), 739-743, which is incorporated herein by reference. At the end of incubation, the reaction mixture was applied on a PD10 column (GE Healthcare Life Sciences) and the product DFO-NCS-CX-2009 (“DFO-CX-2009”) collected in 1 mL of 20 mM L-histidine/240 mM sucrose/0.01% Tween 20. Radiolabeling of DFO-CX-2009 (350 μL) with ⁸⁹Zr (120 MBq) was performed for 60 min at room temperature in a 2 mL reaction at pH 7 using 0.5 M HEPES for buffering. After labeling, the reaction mixture was applied on a PD-10 column and ⁸⁹Zr-DFO-CX-2009 was collected in 2.5 mL 20 mM L-histidine/240 mM sucrose/0.01% Tween 20 (pH 5.4-5.6).

### B. ⁸⁹Zr-CX-191

⁸⁹Zr-CX-191 was prepared analogously to ⁸⁹Zr-CX-2009. Briefly, 2.5 mg of CX-191 (9.4 mg/mL) were diluted to a 5 mg/mL solution with 0.9% NaCl, followed by adjustment of the pH to 8.9-9.1 with 0.1 M Na₂CO₃ and reacted with 3 equivalents of DFO-NCS in DMSO (5 mM, 10 μL) at 37° C. for 30 min. Purification of DFO-CX-191, its radiolabeling with ⁸⁹Zr (85 MBq) in a 2 mL reaction volume, and purification of ⁸⁹Zr-CX-191 were the same as described for ⁸⁹Zr-CX-2009.

### C. ⁸⁹Zr-CX-1031

CX-1031 was first rebuffered. To this end, two mg of CX-1031 (4.3 mg/mL) were diluted to 0.5 mL with 0.9% NaCl and applied on a PD10 column. The product was collected in 1.5 mL 0.9% NaCl. The pH of this solution was adjusted to 8.9-9.1 with 0.1 M Na₂CO₃ and further reacted with 5 equivalents of DFO-NCS in DMSO (5 mM, 13 μL) at 37° C. for 30 min. Purification of DFO-CX-1031, its radiolabeling with ⁸⁹Zr (50 MBq) in a 2 mL reaction volume, and purification of ⁸⁹Zr-CX-1031 were the same as described for ⁸⁹Zr-CX-2009.

### D. ⁸⁹Zr-CX-090

Before radiolabeling CX-090, an additional first step of rebuffering was done. To this end, 3 mg of CX-090 in PBS (13.28 mg/mL) was diluted to 0.5 mL with 0.9% NaCl and applied on a PD10 column. CX-090 was collected in a 1 mL 0.9% NaCl solution and its concentration was determined with Nanodrop. The pH of the CX-090 solution (2.1 mg/mL) was adjusted to 8.9-9.1 with 0.1 M Na₂CO₃, and further reacted with 5 equivalents of DFO-NCS in DMSO (5 mM, 13 μL) at 37° C. for 30 min. Purification of DFO-CX-090, its radiolabeling with ⁸⁹Zr (97 MBq) in a 2 mL reaction volume, and purification of ⁸⁹Zr-CX-090 were the same as described for ⁸⁹Zr-CX-2009.

### Example 3

### Radiochemical Purity and Conjugate Concentration, Integrity and Binding

The radiolabeled products were checked for their radiochemical purity by size-exclusion high performance liquid chromatography (SE-HPLC) and spin filter analysis. A Jasco HPLC system was equipped with a Superdex® 200 Increase 10/300 GL (30 cm×10 mm, 8.6 μm) size exclusion column (GE Healthcare Life Sciences) and a guard column using a 0.05 M phosphate buffer/0.15 M NaCl/0.01 NaN₃ (pH 6.7) as mobile phase with a run time of 40 min at 0.75 mL/min. The radioactivity was monitored with an inline NaI(TI) radiodetector (Raytest Sockett). The radiolabeled antibody constructs eluted at approximately 15 min and ⁸⁹Zr/⁸⁹Zr-chelator at approximately 27 min. The radiochemical purity was expressed as the percentage of the area under peak of the radiolabeled product on the radioactive channel. The radiochemical purity was also assessed by spin filter analysis. To this end, 4 μL of product was diluted with 96 eluent (5% DMSO and 95% 20 mM Histidine/240 mM sucrose buffer/0.01% Tween 20) and applied on a microcon-30 centrifugal filter unit (Ultracel YM-30, regenerated cellulose, 30 kDa cut-off, Merck Millipore). The solution was spun down for 7 min at 14000 rpm (Eppendorf 5430). The filter was washed twice with 100 μl eluent and spun down at 14000 rpm for 7 min after each wash step. The filtrate contained free ⁸⁹Zr/⁸⁹Zr-DFO, while the radiolabeled constructs were left on the filter. Concentration and integrity were assessed on the same SE-HPLC system described above using the areas under curve on the UV channel at 280 nm. The concentration was determined against a calibration curve of the cold compound.

⁸⁹Zr-CX-2009, ⁸⁹Zr-CX-191, and ⁸⁹Zr-CX-090 were efficiently obtained with a radiochemical yield (RCY) of 62%, 70%, and 81%, respectively. ⁸⁹Zr-CX-1031 was obtained with a lower RCY of 32%, but sufficient yield for the in vivo studies. The radiochemical purities assessed by the average of spin filter and HPLC results were above 95% for all constructs.

### Example 4

### Bioactive Agent to Activatable Antibody/Antibody Ratio

The agent conjugate ratio (i.e., ratio of bioactive agent (e.g., DM4) to activatable antibody or antibody) of ⁸⁹Zr-CX-2009 and agent conjugate ratio of ⁸⁹Zr-CX-1031 were determined by HPLC by dividing the area under curve of the PDC/ADC peak at 252 nm by the area under curve of the PDC/ADC peak at 280 nm. A ratio of 0.63±0.10 was determined on cold CX-2009 and CX-1031, being equivalent to an agent conjugate ratio of on average 3.5 and 3.7 DM4 conjugated per molecule, respectively. No DM4 release was observed upon conjugation and radiolabeling.

### Example 5

### Binding Assay

Immunoreactivity of the four radiolabeled constructs was assessed using a CD166 binding assay with radioactive read-out. Extracellular domain CD166 (His-sumo-CD166-ECD) at a concentration of 0.5 mg/mL in PBS+4% trehalose (pH 7.2). One day before production of the radiolabeled constructs, CD166 was diluted in a coating buffer (15 mM sodium carbonate/35 sodium bicarbonate/3 mM sodium azide buffer, pH 9.3-9.8) to a concentration of 5.0 μg/mL and applied to Maxisorp break apart wells (100 μL/well, Thermo Fisher Scientific). After overnight incubation at 4° C., the excess of CD166 antigen was removed and the wells washed three times with PBS (150 μL). Subsequently, the plates were blocked with a solution of 1% BSA/PBS (150 μL) at room temperature while shaking. The plates were then washed three times with a solution of 0.05% Tween 20/PBS (200 μL) before incubation with the radioactive derivatives. As ⁸⁹Zr-CX-2009 and ⁸⁹Zr-CX-191 are masked, a recombinant human protease (matriptase) was used for construct activation prior to incubation in the antigen-coated plates. Without prior “unmasking” of the radiolabeled ⁸⁹Zr-CX-2009 and ⁸⁹Zr-CX-191 with matriptase, both constructs appeared incapable of binding to CD166 (<10% binding).

Ninety microliters of either ⁸⁹Zr-CX-2009 or ⁸⁹Zr-CX-191 at a concentration of 0.5 mg/mL in 20 mM histidine/240 mM sucrose/0.01% Tween 20 were first incubated with 10 μL of the matriptase solution (0.4 mg/mL; specific activity >10,000 pmol/min/μg, R&D systems) for 4 h at 25° C. in a thermomixer without shaking. A serial dilution of the radiolabeled products in 1% BSA/PBS was made in triplicate with a concentration range of 4 μg/mL to 62.5 ng/mL. 100 μL of this solution were added per coated well and incubated overnight at 4° C. while shaking. At the highest dilution, binding was also assessed after addition of 100 μg of cold matriptase-cleaved CX-191 (i.e., comprising cold anti-CD166 antibody) as control for non-specific binding. After 16-24 h, supernatants from each of the wells were collected. Next, the wells were washed three times with 0.05% Tween20/PBS (200 μL) and the washing fractions were pooled with the supernatants. Wells and supernatants were counted separately in a gamma counter (Wallac LKB-CompuGamma 1282; Pharmacia). Immunoreactivity of ⁸⁹Zr-CX-2009, ⁸⁹Zr-CX-191, ⁸⁹Zr-CX-1031 and ⁸⁹Zr-CX-090 was expressed as the percentage of radioactivity bound to the CD166-coated wells compared to the total amount of radioactivity (radiolabeled mAb) added to each well. The results indicated that antigen binding was preserved for all constructs (<70%).

### Example 6

### Ex Vivo Biodistribution Studies

The biodistribution of ⁸⁹Zr-CX-2009, ⁸⁹Zr-CX-191, ⁸⁹Zr-CX-1031, and ⁸⁹Zr-CX-090 was evaluated in H292 tumor bearing mice. After at least one week of acclimation, female nu/nu mice (received at 8 weeks old, Envigo, Harlan ˜18-25 g) were injected subcutaneously (s.c.) in both flanks with 5×10⁶ H292 human lung cancer cells (American Type Culture Collection (ATCC)). Tumor growth was monitored on a daily basis and tumor volume was assessed with a caliper at least twice a week as soon as tumors became detectable. All animal experiments were performed according to the NIH Principles of Laboratory Animal Care and Dutch national law (“Wet op de dierproeven”, Stb 1985, 336). When tumors reached an average volume of ˜200 mm³, mice were randomized and divided in 14 groups of 5 mice for injection with 100-200 μL of the tracers. Injections were performed under anesthesia with inhalation of 2-4% isoflurane/02, intravenously (I.V.) via the retro orbital plexus with either ⁸⁹Zr-CX-2009 (10, 110 or 510 μg), ⁸⁹Zr-CX-191 (10, 110, or 510 μg), ⁸⁹Zr-CX-1031 (110 or 510 μg), or ⁸⁹Zr-CX-090 (10, 110 or 510 μg). At 24 and 48 h post injection (p.i.), blood samples were taken and at 72 h p.i. all mice from those groups were anesthetized, bled, euthanized, and dissected.

Biodistribution of 110 μg ⁸⁹Zr-CX-2009 was also assessed at 24 h and 168 h p.i. For the 168 h p.i. group, blood samples were taken at 24, 48 and 72 h p.i. Finally, in one additional group, the animals received a blocking dose of 500 μg of CX-090 24 h prior injection of 510 μg of ⁸⁹Zr-CX-2009, while blood samples were taken at 24 and 48 h p.i. and the mice were sacrificed at 72 h p.i. All mice were injected with on average 0.7±0.1 MBq except for the group sacrificed at 168 h p.i. that received 2.1±0.0 MBq. For all mice, blood, tumors and organs of interest were collected, weighed, and the amount of radioactivity in each sample was measured in a gamma counter (Wallac LKB-CompuGamma 1282; Pharmacia). Radioactivity uptake was calculated as the percentage of the injected dose per gram of tissue (% ID/g). During animal dissection, some healthy organs (liver lobes, kidneys) and halved tumors were collected and flash frozen. Plasma samples were stored at −20° C. after centrifugation and collection. Those samples were analyzed by Western capillary electrophoresis for assessment of activated and intact CX-2009 and CX-191.

Western Capillary Electrophoresis: Homogenates of H292 xenograft tumor and liver tissue were prepared in Pierce™ IP Lysis Buffer (Thermo Scientific) with added Halt™ Protease Inhibitor Cocktail Kit (Thermo Scientific) using Barocycler (Pressure Biosciences). Protein lysates in IP lysis buffer with HALT protease inhibitor/EDTA were analyzed by the Western capillary electrophoresis (Wes™ system, ProteinSimple). Plasma was diluted 1:50 in PBS before analysis on the Wes™ system. Activated and intact CX-2009 and CX-191 were detected using an anti-idiotypic antibody and anti-rat secondary antibody Fc (Jackson ImmunoResearch). The concentration of activated and intact CX-2009 and CX-191 was calculated from the respective standard curves using the Compass software (ProteinSimple) and the method described in PCT publication WO 2019/018828 A1, which is incorporated herein by reference.

Statistics: The Grubbs outlier test was used to check and remove outliers and statistical analysis was performed on the tissue uptake values of the different groups of mice with the Welch's T-test for paired data. Two-sided significance levels were calculated and p>0.05 was considered to be statistically significant. All graphs were generated using GraphPad Prism 5.02 software.

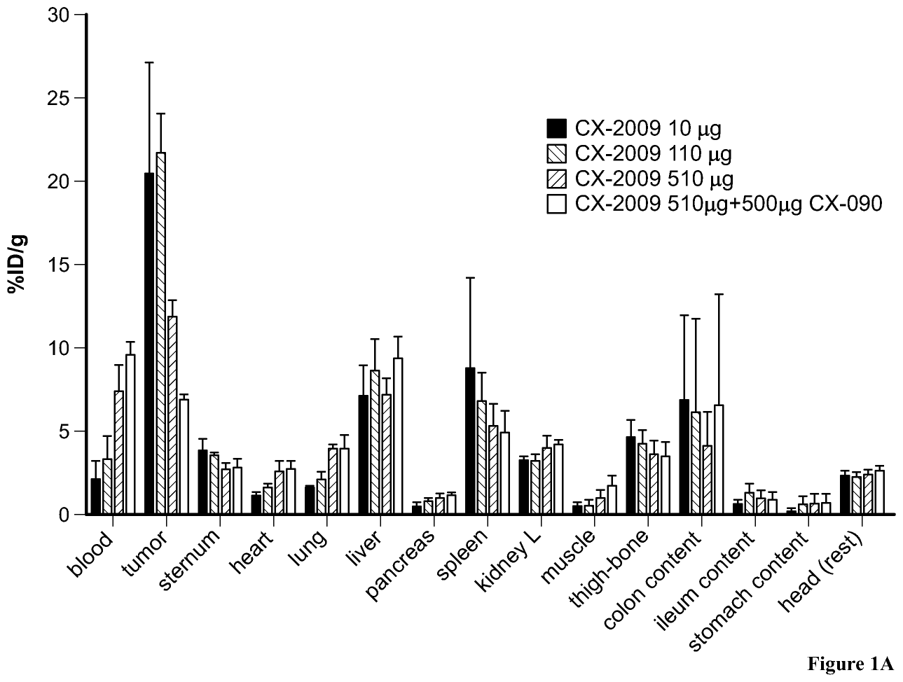

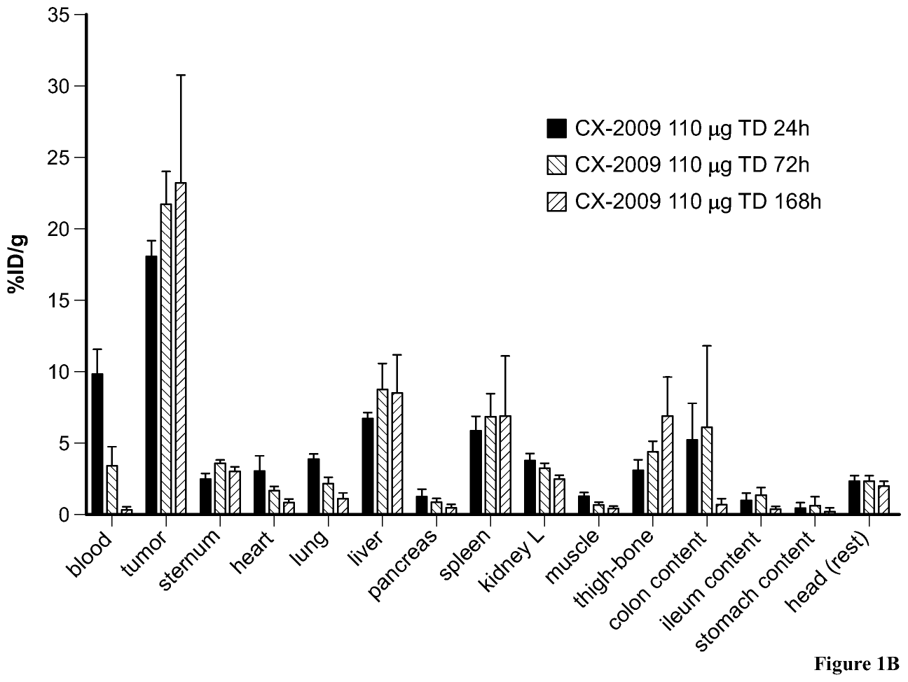

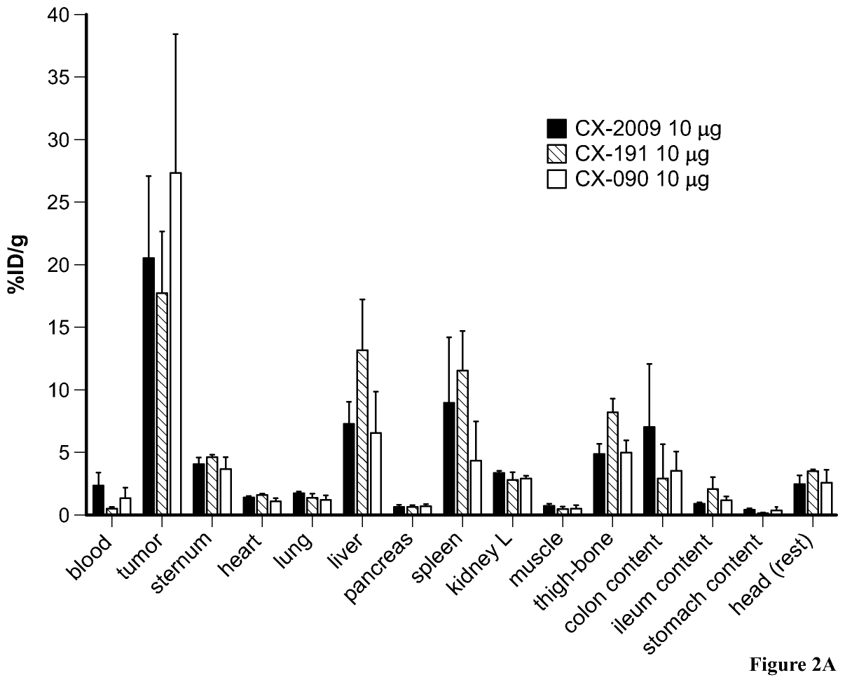

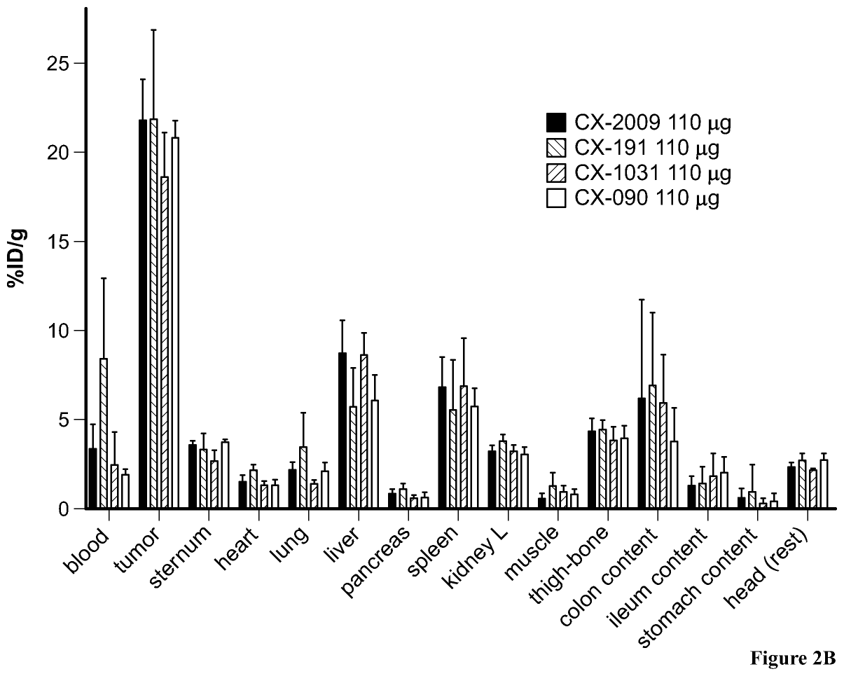

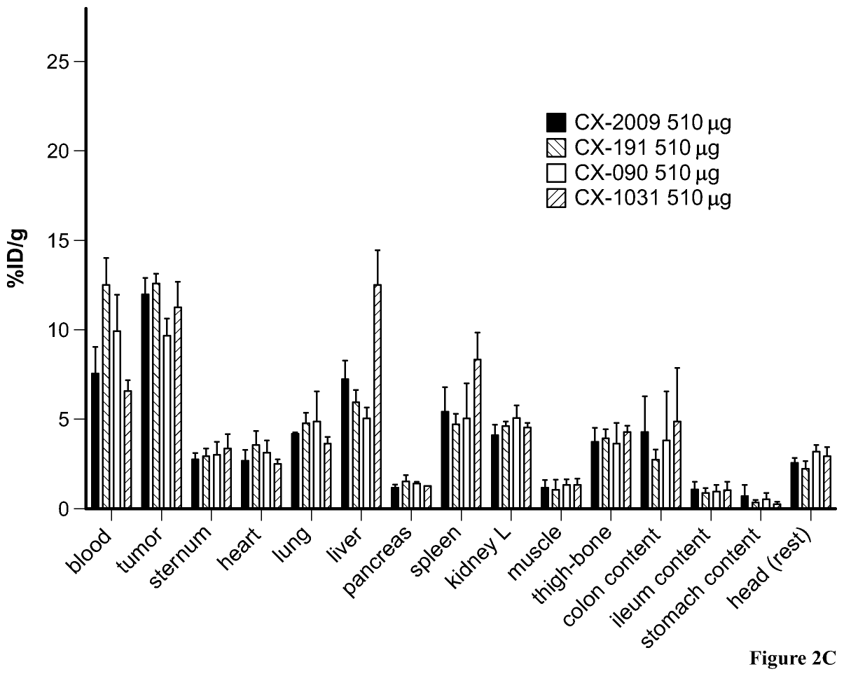

Results: The biodistribution of ⁸⁹Zr-CX-2009 was assessed as a function of dose (10, 110, or 510 μg), as depicted in FIG. 1A and time (24, 72 and 168 h p.i., as depicted in FIG. 1B. Highest ⁸⁹Zr-CX-2009 tumor uptake of 20.5±6.6% ID/g and 21.8±2.3% ID/g at 72 h p.i. was observed for the 10 and 110 μg groups, with lower standard deviations for the 110 μg group. Tumor uptake values for these two groups were much higher than the blood values of 2.2±1.1% ID/g and 3.4±1.3% ID/g, respectively. Increasing the dose with unlabeled CX-2009 from 10 μg (FIG. 2A) to saturating levels, 510 μg (FIG. 2C) was associated with lower tumor uptake and higher blood values for all constructs while increased variation in tumor uptake within and between groups was observed at 10 μg. Optimal tumor targeting was obtained with 110 μg. See FIG. 2B. At 72 h p.i. ⁸⁹Zr-CX-2009 (110 μg dose) presented a tumor uptake of 21.8±2.3% ID/g, which was not significantly different in comparison with ⁸⁹Zr-CX-191 (21.8±5.0), 89Zr-CX-1031 (18.7±2.5), and ⁸⁹Zr-CX-090 (20.8±0.9% ID/g), as shown in FIG. 2B. Tumor uptake of ⁸⁹Zr-CX-2009 (110 μg dose) slightly increased in time from 18.0±1.2 at 24 h p.i. to 21.8±2.3 at 72 h p.i. (p<0.05) and 23.5±7.3% ID/g at 168 h p.i. while blood levels steadily decreased over this time period, as shown in FIG. 1B.

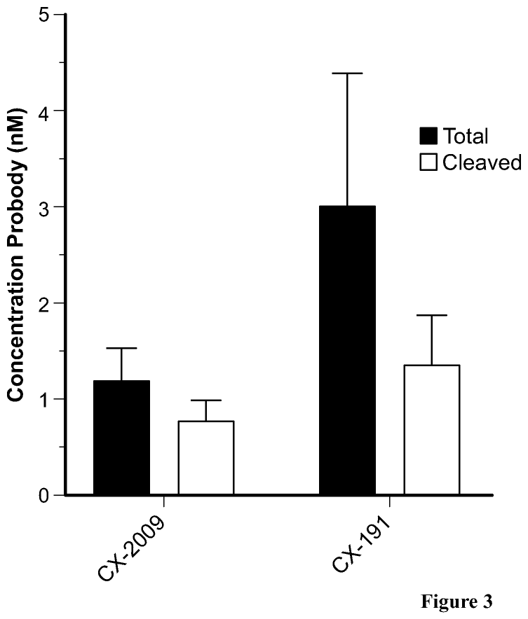

Concentration of total and activated CX-2009 and CX-191 constructs was measured in H292 tumor tissues collected 72 h after tracer administration using Western capillary electrophoresis method. The corresponding activation rate of 67% and 46% was detected for CX-2009 and CX-191, respectively, as shown in FIG. 3.

### B. PET Imaging Studies

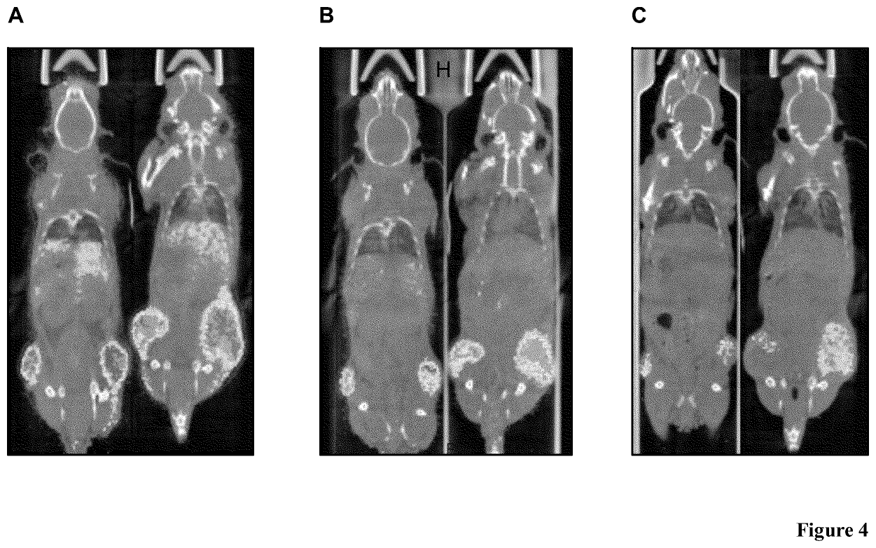

PET imaging was performed on a dedicated small animal Nano/PET/CT scanner (Mediso Ltd., Hungary, Szanda, et al.). Four mice from each of the groups that received 110 μg of either ⁸⁹Zr-CX-2009, ⁸⁹Zr-CX-191, ⁸⁹Zr-CX-1031, or ⁸⁹Zr-CX02009 were imaged at 24 h and 72 h p.i. with additional imaging at 168 h p.i. for ⁸⁹Zr-CX-2009. Mice were anesthetized by inhalation of 2-4% isoflurane/O2 during the whole scanning period (1 h duration per time point). A 5 min CT scan was acquired prior to each PET scan and used for attenuation and scatter correction purposes. Reconstruction was performed by 3-dimensional (3-D) reconstruction (TeraTomo; Mediso Ltd.) with four iterations and six subsets, resulting in an isotropic 0.4 mm voxel dimension. The scanner was cross-calibrated with the dose-calibrator and well counter, enabling accurate measurement of Standard Uptake Values (SUVs). SUV values were calculated as the ratio of the radioactivity activity concentration (MBq/mL) measured by the PET scanner within the region of interest (ROI), divided by the decay-corrected amount of injected radiolabeled compound corrected for the weight of the animal. The software Amide (GNU General Public License, Version 2. Made.exe 0.9.2) was used to draw and quantify the ROIs and VivoQuant to capture images and videos displayed. Examples of mice injected with 110 μg of ⁸⁹Zr-CX-2009 scanned over time are presented in FIG. 4.

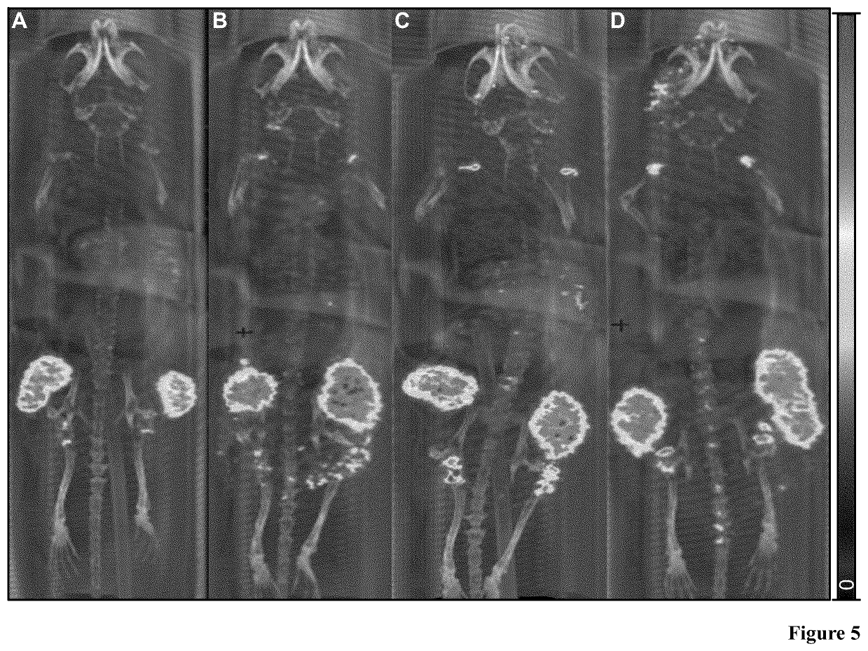

Quantitative PET imaging confirmed the similar uptake of the four constructs in the tumors. At 72 h p.i. and a dose of 110 μg, SUVs of the tumors remained similar with 4.8±0.2 for ⁸⁹Zr-CX-2009, 4.2±0.4 for ⁸⁹Zr-CX-191, 4.4±0.4 for ⁸⁹Zr-CX-1031 and 4.4±0.4 for ⁸⁹Zr-CX-090 (FIG. 5). Finally, the same mice from the 100 μg ⁸⁹Zr-CX-2009 group imaged at 168 h p.i. presented a tendency to a higher uptake in the tumor with a SUV of 5.6±1.9.

TABLE 1
Table of Sequences

| SEQ ID NO: | NAME | SEQUENCE |
|---|---|---|
| 1 | CM | LSGRSDNH |
| 2 | CM | TGRGPSWV |
| 3 | CM | PLTGRSGG |
| 4 | CM | TARGPSFK |
| 5 | CM | NTLSGRSENHSG |
| 6 | CM | NTLSGRSGNHGS |
| 7 | CM | TSTSGRSANPRG |
| 8 | CM | TSGRSANP |
| 9 | CM | VHMPLGFLGP |
| 10 | CM | AVGLLAPP |
| 11 | CM | AQNLLGMV |
| 12 | CM | QNQALRMA |
| 13 | CM | LAAPLGLL |
| 14 | CM | STFPFGMF |
| 15 | CM | ISSGLLSS |
| 16 | CM | PAGLWLDP |
| 17 | CM | VAGRSMRP |
| 18 | CM | VVPEGRRS |
| 19 | CM | ILPRSPAF |
| 20 | CM | MVLGRSLL |
| 21 | CM | QRAITFI |
| 22 | CM | SPRSIMLA |
| 23 | CM | SMLRSMPL |
| 24 | CM | ISSGLLSGRSDNH |
| 25 | CM | AVGLLAPPGGLSGRSDNH |
| 26 | CM | ISSGLLSSGGSGGSLSGRSDNH |
| 27 | CM | LSGRSGNH |
| 28 | CM | SGRSANPRG |
| 29 | CM | LSGRSDDH |
| 30 | CM | LSGRSDIH |
| 31 | CM | LSGRSDQH |
| 32 | CM | LSGRSDTH |
| 33 | CM | LSGRSDYH |
| 34 | CM | LSGRSDNP |
| 35 | CM | LSGRSANP |
| 36 | CM | LSGRSANI |
| 37 | CM | LSGRSDNI |
| 38 | CM | MIAPVAYR |
| 39 | CM | RPSPMWAY |
| 40 | CM | WATPRPMR |
| 41 | CM | FRLLDWQW |
| 42 | CM | ISSGL |
| 43 | CM | ISSGLLS |
| 44 | CM | ISSGLL |
| 45 | CM | ISSGLLSGRSANPRG |
| 46 | CM | AVGLLAPPTSGRSANPRG |
| 47 | CM | AVGLLAPPSGRSANPRG |
| 48 | CM | ISSGLLSGRSDDH |
| 49 | CM | ISSGLLSGRSDIH |
| 50 | CM | ISSGLLSGRSDQH |
| 51 | CM | ISSGLLSGRSDTH |
| 52 | CM | ISSGLLSGRSDYH |
| 53 | CM | ISSGLLSGRSDNP |
| 54 | CM | ISSGLLSGRSANP |
| 55 | CM | ISSGLLSGRSANI |
| 56 | CM | AVGLLAPPGGLSGRSDDH |
| 57 | CM | AVGLLAPPGGLSGRSDIH |
| 58 | CM | AVGLLAPPGGLSGRSDQH |
| 59 | CM | AVGLLAPPGGLSGRSDTH |
| 60 | CM | AVGLLAPPGGLSGRSDYH |
| 61 | CM | AVGLLAPPGGLSGRSDNP |
| 62 | CM | AVGLLAPPGGLSGRSANP |
| 63 | CM | AVGLLAPPGGLSGRSANI |
| 64 | CM | ISSGLLSGRSDNI |
| 65 | CM | AVGLLAPPGGLSGRSDNI |
| 66 | CM | GLSGRSDNHGGAVGLLAPP |
| 67 | CM | GLSGRSDNHGGVHMPLGFLGP |
| 68 | Linker | GSGGS |
| 69 | Linker | GGGS |
| 70 | Linker | GGSG |
| 71 | Linker | GGSGG |
| 72 | Linker | GSGSG |
| 73 | Linker | GSGGG |
| 74 | Linker | GGGSG |
| 75 | Linker | GSSSG |
| 76 | Linker | GSSGGSGGSGGSG |
| 77 | Linker | GSSGGSGGSGG |
| 78 | Linker | GSSGGSGGSGGS |
| 79 | Linker | GSSGGSGGSGGSGGGS |
| 80 | Linker | GSSGGSGGSG |
| 81 | Linker | GSSGGSGGSGS |
| 82 | Linker | GSSGT |
| 83 | Linker | GSSG |
| 84 | MM | LCHPLVLSAWESCSS |
| 85 | MM | LCHPAVLSAWESCSS |
| 86 | MM | LCHPLVASAWESCSS |
| 87 | MM | LEGWCLHPLCLWGAG |
| 88 | MM | LCAPLVLSAWESCSS |
| 89 | MM | LCHALVLSAWESCSS |
| 90 | MM | LCHPLALSAWESCSS |
| 91 | MM | LCHPLVLSAAESCSS |
| 92 | MM | LCHPLVLSAWASCSS |
| 93 | MM | HPLVL |
| 94 | MM | LEGACLHPLCLWGAG |
| 95 | MM | LEGWCAHPLCLWGAG |
| 96 | MM | LEGWCLAPLCLWGAG |
| 97 | MM | LEGWCLHACLWGAG |
| 98 | MM | LEGWCLHPACLWGAG |
| 99 | MM | LEGWCLHPLCAWGAG |
| 100 | MM | LEGWCLHPLCLAGAG |
| 101 | MM | CLHPLC |
| 102 | Spacer | QGQSGQ |
| 103 | Spacer | QGQSGQG |
| 104 | Spacer | QGQSG |
| 105 | Spacer | QGQS |
| 106 | Spacer | GQSGQG |
| 107 | Spacer | QSGQG |
| 108 | Spacer | SGQG |
| 109 | Spacer | GQSGQG |
| 110 | Spacer | QSGQG |
| 111 | Spacer | SGQG |
| 112 | VH CDR1 | GFSLSTYGMGVG |
| 113 | VH CDR2 | NIWWSEDKH |
| 114 | VH CDR3 | IDYGNDYAFTY |
| 115 | VL CDR1 | RSSKSLLHSNGITYLY |
| 116 | VL CDR2 | QMSNLAS |
| 117 | VL CDR3 | AQNLELPYT |
| 118 | VH | QITLKESGPTLVKPTQTLTLTCTFSGFSLSTYGMGVGWIRQPPGKALE |
| | | WLANIWWSEDKHYSPSLKSRLTITKDTSKNQVVLTMTNMDPVDTATYY |
| | | CVQIDYGNDYAFTYWGQGTLVTVSS |
| 119 | VH | QITLKESGPTLVKPTQTLTLTCTFSGFSLSTYGMGVGWIRQPPGKALE |
| | | WLANIWWSEDKHYSPSLKSRLTITKDTSKNQVVLTITNVDPVDTATYY |
| | | CVQIDYGNDYAFTYWGQGTLVTVSS |
| 120 | VL | DIVMTQSPLSLPVTPGEPASISCRSSKSLLHSNGITYLYWYLQKPGQS |
| | | PQLLIYQMSNLASGVPDRFSGSGSGTDFTLKISRVEAEDVGVYYCAQN |
| | | LELPYTFGQGTKLEIK |
| 121 | VL | DIVMTQSPLSLPVTPGEPASISCRSSKSLLHSNGITYLYWYLQKPGQS |
| | | PQLLIYQMSNLASGVPDRFSSSGSGTDFTLKISRVEAEDVGVYYCAQN |
| | | LELPYTFGQGTKLEIK |
| 122 | VL | DIVMTQSPLSLPVTPGEPASISCRSSQSLLHSNGITYLYWYLQKPGQS |
| | | PQLLIYQMSNRASGVPDRFSGSGSGTDFTLKISRVEAEDVGVYYCAQN |
| | | LELPYTFGQGTKLEIK |
| 123 | VL | DIVMTQSPLSLPVTPGEPASISCRSSQSLLHSNGITYLYWYLQKPGQS |
| | | PQLLIYQMSNRASGVPDRFSSSGSGTDFTLKISRVEAEDVGVYYCAQN |
| | | LELPYTFGQGTKLEIK |
| 124 | VL CDR1 | RSSQSLLHSNGITYLY |
| 125 | VL CDR2 | QMSNRAS |
| 126 | Heavy Chain | QITLKESGPTLVKPTQTLTLTCTFSGFSLSTYGMGVGWIRQPPGKALE |
| | (activatable | WLANIWWSEDKHYSPSLKSRLTITKDTSKNQVVLTITNVDPVDTATYY |
| | anti-huCD166) | CVQIDYGNDYAFTYWGQGTLVTVSSASTKGPSVFPLAPSSKSTSGGTA |
| | | ALGCLVKDYFPEPVTVSWNSGALTSGVHTFPAVLQSSGLYSLSSVVTV |
| | | PSSSLGTQTYICNVNHKPSNTKVDKKVEPKSCDKTHTCPPCPAPELLG |
| | | GPSVFLFPPKPKDTLMISRTPEVTCVVVDVSHEDPEVKFNWYVDGVEV |
| | | HNAKTKPREEQYNSTYRVVSVLTVLHQDWLNGKEYKCKVSNKALPAPI |
| | | EKTISKAKGQPREPQVYTLPPSREEMTKNQVSLTCLVKGFYPSDIAVE |
| | | WESNGQPENNYKTTPPVLDSDGSFFLYSKLTVDKSRWQQGNVFSCSVM |
| | | HEALHNHYTQKSLSLSPG |
| 127 | Light Chain | QGQSGQGLCHPAVLSAWESCSSGGGSSGGSAVGLLAPPGGLSGRSDNH |
| | (activatable | GGSDIVMTQSPLSLPVTPGEPASISCRSSKSLLHSNGITYLYWYLQKP |
| | anti-huCD166) | GQSPQLLIYQMSNLASGVPDRFSGSGSGTDFTLKISRVEAEDVGVYYC |
| | | AQNLELPYTFGQGTKLEIKRTVAAPSVFIFPPSDEQLKSGTASVVCLL |
| | | NNFYPREAKVQWKVDNALQSGNSQESVTEQDSKDSTYSLSSTLTLSKA |
| | | DYEKHKVYACEVTHQGLSSPVTKSFNRGEC |
| 128 | Linker | GGGSSGGSGGSGG |
| 129 | Spacer | QGQSGS |
| 130 | Spacer | GQSGS |
| 131 | Spacer | QSGS |
| 132 | Spacer | GQSGQ |
| 133 | Spacer | QSGQ |
| 134 | huCD166 | MESKGASSCRLLFCLLISATVFRPGLGWYTVNSAYGDTIIIPCRLDVP |
| | | QNLMFGKWKYEKPDGSPVFIAFRSSTKKSVQYDDVPEYKDRLNLSENY |
| | | TLSISNARISDEKRFVCMLVTEDNVFEAPTIVKVFKQPSKPEIVSKAL |
| | | FLETEQLKKLGDCISEDSYPDGNITWYRNGKVLHPLEGAVVIIFKKEM |
| | | DPVTQLYTMTSTLEYKTTKADIQMPFTCSVTYYGPSGQKTTHSEQAVF |
| | | DIYYPTEQVTIQVLPPKNAIKEGDNITLKCLGNGNPPPEEFLFYLPGQ |
| | | PEGIRSSNTYTLMDVRRNATGDYKCSLIDKKSMIASTAITVHYLDLSL |
| | | NPSGEVTRQIGDALPVSCTISASRNATVVWMKDNIRLRSSPSFSSLHY |
| | | QDAGNYVCETALQEVEGLKKRESLTLIVEGKPQIKMTKKTDPSGLSKT |
| | | TICHVEGFPKPAIQWTITGSGSVINQTEESPYINGRYYSKIIISPEEN |
| | | VTLTCTAENQLERTVNSLNVSAISIPEHDEADEISDENREKVNDQAKL |
| | | IVGIVVGLLLAALVAGVVYWLYMKKSKTASKHVNKDLGNMEENKKLEE |
| | | NNHKTEA |

While the foregoing invention has been described in some detail for purposes of clarity and understanding, it will be clear to one skilled in the art from a reading of this disclosure that various changes in form and detail can be made without departing from the true scope of the invention. It is understood that the materials, examples, and embodiments described herein are for illustrative purposes only and not intended to be limiting and that various modifications or changes in light thereof will be suggested to persons skilled in the art and are to be included within the spirit and scope of the appended claims.

We claim:

1. A method for detecting an in vivo distribution of a radiolabeled activated activatable anti-CD166 antibody-agent conjugate in a subject, the method comprising:
administering to a subject a tracer dose of a radiolabeled activatable anti-CD166 antibody-agent conjugate,
wherein the radiolabeled activatable anti-CD166 antibody-agent conjugate comprises a radionuclide coupled to an activatable anti-CD166 antibody-agent conjugate,
wherein the activatable anti-CD166 antibody-agent conjugate comprises
(i) an anti-CD166 antibody or an antigen binding fragment thereof (AB) that specifically binds to a mammalian CD166;
(ii) a prodomain comprising a masking moiety (MM) and a cleavable moiety (MM), wherein the prodomain is coupled, either directly or indirectly, to the AB; and
(iii) a bioactive agent conjugated to the AB,
wherein, when the radiolabeled activatable anti-CD166 antibody-agent conjugate is activated, a corresponding radiolabeled activated activatable anti-CD166 antibody-agent conjugate is generated that is capable of specifically binding the mammalian CD166; and
imaging the subject using positron emission tomography (PET) at a time point following administration of the tracer dose to detect the presence of the radionuclide, thereby detecting the in vivo distribution of radiolabeled activated activatable anti-CD166 antibody-agent conjugate in the subject.

2. The method of claim 1, wherein the AB comprises:
(a) a variable heavy chain complementarity determining region 1 (VH CDR1) comprising the amino acid sequence of SEQ ID NO:112;
(b) a variable heavy chain complementarity determining region 2 (VH CDR2) comprising the amino acid sequence of SEQ ID NO:113;
(c) a variable heavy chain complementarity determining region 3 (VH CDR3) comprising the amino acid sequence of SEQ ID NO:114;
(d) a variable light chain complementarity determining region 1 (VL CDR1) comprising the amino acid sequence of SEQ ID NO:115;
(e) a variable light chain complementarity determining region 2 (VL CDR2) comprising the amino acid sequence of SEQ ID NO:116;
(f) a variable light chain complementarity determining region 3 (VL CDR3) comprising the amino acid sequence of SEQ ID NO:117.

3. The method of claim 1, wherein the AB comprises:
(a) a variable heavy chain complementarity determining region 1 (VH CDR1) comprising the amino acid sequence of SEQ ID NO:112;
(b) a variable heavy chain complementarity determining region 2 (VH CDR2) comprising the amino acid sequence of SEQ ID NO:113;
(c) a variable heavy chain complementarity determining region 3 (VH CDR3) comprising the amino acid sequence of SEQ ID NO:114;
(d) a variable light chain complementarity determining region 1 (VL CDR1) comprising the amino acid sequence of SEQ ID NO:124;
(e) a variable light chain complementarity determining region 2 (VL CDR2) comprising the amino acid sequence of SEQ ID NO:125;
(f) a variable light chain complementarity determining region 3 (VL CDR3) comprising the amino acid sequence of SEQ ID NO:117.

4. The method of claim 1, wherein the AB comprises a heavy chain variable region comprising an amino acid sequence selected from the group consisting of SEQ ID NO:118 and SEQ ID NO:119, and a light chain variable region comprising an amino acid sequence selected from the group consisting of SEQ ID NO:120, SEQ ID NO:121, SEQ ID NO:122, and SEQ ID NO:123.

5. The method of any one of claims 1-2, wherein the AB comprises a heavy chain variable region (VH) comprising the amino acid sequence of SEQ ID NO:119 and a light chain variable region (VL) comprising the amino acid sequence of SEQ ID NO:120.

6. The method of any one of claims 1-5, wherein the prodomain comprises an MM that comprises an amino acid sequence selected from the group consisting of any one of SEQ ID NOs:84-99 and HPL.

7. The method of any one of claims 1-6, wherein the prodomain comprises a CM that comprises an amino sequence selected from the group consisting of any one of SEQ ID NOs:1-67.

8. The method of any one of claims 1-7, wherein the prodomain comprises a spacer comprising an amino acid sequence selected from the group consisting of any one of SEQ ID NOs:102-111 and 129-133.

9. The method of any one of claims 1-8, wherein the prodomain is linked indirectly to the AB via a linker comprising an amino acid sequence selected from the group consisting of any one of SEQ ID NOs:69-83, 128, SGS, GS, S, GQG, QG, G, SGQ, GQ, and Q.

10. The method of any one of claims 1-9, wherein the MM and CM of the prodomain are coupled indirectly to each other via a linker having an amino acid sequence selected from the group consisting of any one of SEQ ID NOs:69-83, 128, SGS, GS, S, GQG, QG, G, SGQ, GQ, and Q.

11. The method of any one of claims 1-2, wherein the radiolabeled activatable anti-CD166 antibody-agent conjugate comprises a light chain and a heavy chain,
wherein the light chain comprises the prodomain and a VL, and wherein the light chain comprises the amino acid sequence of SEQ ID NO:127; and
wherein the heavy chain comprises the amino acid sequence of SEQ ID NO:126.

12. The method of any one of claims 1-11, wherein the bioactive agent comprises a cytotoxic agent.

13. The method of claim 12, wherein the cytotoxic agent is selected from the group consisting of an auristatin, a dolastatin, a maytansinoid, a duocarmycin, an amanitin, an anthracycline, doxorubicin, caunorubicin, a bryostatin, a camptothecin, a combretastatin, a debromoaplysiatoxin, kahalalide-F, discodermolide, an ecteinascidins, a turbostatin, a phenstatin, a spongistatin, a halistatin, a bryostatin, a halocomstatin, a pyrrolobenzimidazole, cibrostatin6, doxaliform, an anthracycline, a cemadotin, a Pseudomonas toxin A, a superstolide A, a saponin, an O6-benzylguanine, a topoiosomerase inhibitor, a hemiasterlin, a cephalotaxine, a hemoharringtonine, a pyrrolobenzodiazepene, a calicheamicin, a podophyllotoxin, a taxane, and a vinca alkaloid.

14. The method of any one of claims 1-11, wherein the bioactive agent comprises an antiviral agent.

15. The method of claim 14, wherein the antiviral agent is selected from the group consisting of acyclovir, Vira A, and Symmetrel.

16. The method of any one of claims 1-11, wherein the bioactive agent comprises an antifungal agent.

17. The method of any one of claims 1-11, wherein the bioactive agent comprises an anti-neoplastic agent.

18. The method of any one of claims 1-11, wherein the bioactive agent comprises a heavy metal.

19. The method of any one of claims 1-11, wherein the bioactive agent comprises an anti-bacterial agent.

20. The method of any one of claims 1-11, wherein the bioactive agent comprises an anti-mycoplasmal agent.

21. The method of any one of claims 1-20, wherein the bioactive agent is conjugated to the activatable anti-CD166 antibody via a conjugation linker.

22. The method of any one of claims 1-21, wherein the radionuclide is selected from the group consisting of ¹¹¹In, ¹³¹I, ¹²³I, ^{99m}Tc, ¹⁷⁷Lu, ⁸⁹Zr, ¹²⁴I, ⁵³Cu, ⁸⁶Y, ⁷⁰Br, ¹⁸F, and ⁶⁸Ga.

23. The method of claim 22, wherein the radionuclide is ⁸⁹Zr.

24. The method of any one of claims 1-23, wherein the radionuclide is coupled to the activatable anti-CD166 antibody-agent conjugate via a chelation moiety.

25. The method of claim 24, wherein the chelation moiety comprises a structure corresponding to a chelation agent selected from the group consisting of diethylenetriaminepentaacetic acid, ethylenediaminetetraacetic acid, 1,4,7,10-tetraacetic acid, and desferrioxamine (DFO).

26. The method of claim 25, wherein the chelation moiety comprises a structure corresponding to desferrioxamine (DFO).

27. The method of any one of claims 1-2,
wherein the radiolabeled activatable anti-CD166 antibody-agent conjugate comprises a light chain and a heavy chain,
wherein the light chain comprises the prodomain and a VL, and wherein the light chain comprises the amino acid sequence of SEQ ID NO:127;
wherein the heavy chain comprises the amino acid sequence of SEQ ID NO:126;
wherein the bioactive agent comprises DM4, and
wherein the radionuclide comprises ⁸⁹Zr.

28. The method of claim 27, wherein the radionuclide is coupled to the radiolabeled activatable anti-CD166 antibody-agent conjugate via a chelation moiety having a structure corresponding to desferrioxamine.

29. The method of any one of claims 1-28, wherein the radiolabeled activatable anti-CD166 antibody-agent conjugate comprises two identical light chains and two identical heavy chains.

30. The method of any one of claims 1-29, further comprising administering a blocking dose to the subject, wherein the blocking dose comprises a corresponding non-radiolabeled compound selected from the group consisting of a corresponding non-radiolabeled activatable anti-CD166 antibody-agent conjugate and a corresponding non-radiolabeled activatable anti-CD166 antibody.

31. The method of claim 30, wherein the blocking dose comprises a corresponding non-radiolabeled activatable anti-CD166 antibody-agent conjugate.

32. The method of claim 27, wherein administration of the blocking dose precedes administration of the tracer dose.

33. The method of any one of claims 30-32, wherein the blocking dose comprises from about 0.25 mg/kg to about 10 mg/kg, or from about 0.25 mg/kg to about 6 mg/kg, or from about 6 mg/kg to about 10 mg/kg of the corresponding non-radiolabeled activatable anti-CD166 antibody-agent conjugate.

34. The method of any one of claims 1-33, wherein the tracer dose comprises 37 MBq of the radiolabeled activatable anti-CD166 antibody-agent conjugate.

35. The method of any one of claims 1-34, wherein the imaging step occurs at a time point in the period of from about 1 day to about 10 days post tracer dose administration, or at a time point in the period of from about 2 days to about 10 days post tracer dose administration, or in the period of from about 2 days to about 9 days post tracer dose administration, or in the period of from about 2 days to about 8 days post tracer dose administration, or in the period of from about 3 days to about 10 days post tracer dose administration, or in the period from about 3 days to about 9 days post tracer dose administration, or in the period of from about 3 days to about 8 days post tracer dose administration.

36. The method of any one of claims 1-35, wherein the imaging step occurs at a time point in the period of from about 1 day to about 10 days post tracer dose administration.

37. The method of any one of claims 1-35, wherein the imaging step occurs at a time point in the period of from about 2 days to about 10 days post tracer dose administration.

38. The method of any one of claims 1-35, wherein the imaging step occurs at a time point in the period of from about 2 days to about 9 days post tracer dose administration.

39. The method of any one of claims 1-35, wherein the imaging step occurs at a time point in the period of from about 2 days to about 8 days post tracer dose administration.

40. The method of any one of claims 1-35, wherein the imaging step occurs at a time point in the period of from about 3 days to about 10 days post tracer dose administration.

41. The method of any one of claims 1-35, wherein the imaging step occurs at a time point in the period of from about 3 days to about 9 days post tracer dose administration.

42. The method of any one of claims 1-35, wherein the imaging step occurs at a time point in the period of from about 3 days to about 8 days post tracer dose administration.

43. The method of any one of claims 1-42, wherein the mammalian CD166 is a human CD166.

44. The method of any one of claims 1-43, wherein the subject is a human.

45. The method of any one of claims 1-44, wherein the subject has a cancer.

46. The method of claim 45, wherein the subject has a solid tumor.

47. A method for identifying a subject suitable for treatment with an activatable anti-CD166 antibody-agent conjugate, the method comprising:
detecting the in vivo distribution of a radiolabeled activated activatable anti-CD166 antibody-agent conjugate in accordance with the method of any one of claims 1-42 in a subject having a tumor; and
identifying the subject as being suitable for treatment with a corresponding non-radiolabeled activatable anti-CD166 antibody-agent conjugate if the radionuclide is detectably present within the PET image of the tumor.

48. The method of claim 47, wherein the subject is a human and the mammalian CD166 is a human CD166.

49. The method of any one of claims 47-48, further comprising obtaining a tumor tissue sample from the subject.

50. A method of treating a subject with an activatable anti-CD166 antibody-agent conjugate, the method comprising:
identifying a subject suitable for treatment with an activatable anti-CD166 antibody-agent conjugate in accordance with the method of any one of claims 47-49; and
administering to the subject a therapeutically effective dose of a corresponding non-radiolabeled activatable anti-CD166 antibody-agent conjugate.

51. A ⁸⁹Zr-labeled activatable anti-CD166 antibody-agent conjugate comprising:
⁸⁹Zr coupled via a chelation moiety to an activatable anti-CD166 antibody-agent conjugate, wherein the activatable anti-CD166 antibody-agent comprises
(i) an anti-CD166 antibody or an antigen binding fragment thereof (AB) that specifically binds to a human CD166;
(ii) a prodomain comprising a masking moiety (MM) and a cleavable moiety (MM), wherein the prodomain is coupled, either directly or indirectly, to the AB; and
(iii) a bioactive agent conjugated to the AB,
wherein, when the ⁸⁹Zr-labeled activatable anti-CD166 antibody-agent conjugate is activated, a corresponding ⁸⁹Zr-labeled activated activatable anti-CD166 antibody-agent conjugate is generated that is capable of specifically binding to human CD166.

52. The ⁸⁹Zr-labeled activatable anti-CD166 antibody-agent conjugate of claim 51, wherein the chelation moiety comprises a structure corresponding to desferrioxamine.

53. The ⁸⁹Zr-labeled activatable anti-CD166 antibody-agent conjugate of any one of claims 51-52, wherein the AB comprises:
(a) a variable heavy chain complementarity determining region 1 (VH CDR1) comprising the amino acid sequence of SEQ ID NO:112;
(b) a variable heavy chain complementarity determining region 2 (VH CDR2) comprising the amino acid sequence of SEQ ID NO:113;
(c) a variable heavy chain complementarity determining region 3 (VH CDR3) comprising the amino acid sequence of SEQ ID NO:114;
(d) a variable light chain complementarity determining region 1 (VL CDR1) comprising the amino acid sequence of SEQ ID NO:115;
(e) a variable light chain complementarity determining region 2 (VL CDR2) comprising the amino acid sequence of SEQ ID NO:116; and
(f) a variable light chain complementarity determining region 3 (VL CDR3) comprising the amino acid sequence of SEQ ID NO:117.

54. The ⁸⁹Zr-labeled activatable anti-CD166 antibody-agent conjugate of any one of claims 51-52, wherein the AB comprises:
(a) a variable heavy chain complementarity determining region 1 (VH CDR1) comprising the amino acid sequence of SEQ ID NO:112;
(b) a variable heavy chain complementarity determining region 2 (VH CDR2) comprising the amino acid sequence of SEQ ID NO:113;
(c) a variable heavy chain complementarity determining region 3 (VH CDR3) comprising the amino acid sequence of SEQ ID NO:114;
(d) a variable light chain complementarity determining region 1 (VL CDR1) comprising the amino acid sequence of SEQ ID NO:124;
(e) a variable light chain complementarity determining region 2 (VL CDR2) comprising the amino acid sequence of SEQ ID NO:125; and
(f) a variable light chain complementarity determining region 3 (VL CDR3) comprising the amino acid sequence of SEQ ID NO:117.

55. The ⁸⁹Zr-labeled activatable anti-CD166 antibody-agent conjugate of any one of claims 51-52, wherein the AB comprises a heavy chain variable region comprising an amino acid sequence selected from the group consisting of SEQ ID NO:118 and SEQ ID NO:119, and a light chain variable region comprising an amino acid sequence selected from the group consisting of SEQ ID NO:120, SEQ ID NO:121, SEQ ID NO:122, and SEQ ID NO:123.

56. The ⁸⁹Zr-labeled activatable anti-CD166 antibody-agent conjugate of any one of claims 51-52, wherein the AB comprises a heavy chain variable region comprising the amino acid sequence of SEQ ID NO:119 and a light chain variable region comprising the amino acid sequence of SEQ ID NO:120.

57. The ⁸⁹Zr-labeled activatable anti-CD166 antibody-agent conjugate of any one of claims 51-56, wherein the prodomain comprises an MM that comprises an amino acid sequence selected from the group consisting of any one of SEQ ID NOs:84-101 and HPL.

58. The ⁸⁹Zr-labeled activatable anti-CD166 antibody-agent conjugate of any one of claims 51-57, wherein the prodomain comprises a CM that comprises an amino sequence selected from the group consisting of any one of SEQ ID NOs:1-67.

59. The ⁸⁹Zr-labeled activatable anti-CD166 antibody-agent conjugate of any one of claims 51-58, wherein the prodomain comprises a spacer comprising an amino acid sequence selected from the group consisting of any one of SEQ ID NOs:102-111 and 129-133.

60. The ⁸⁹Zr-labeled activatable anti-CD166 antibody-agent conjugate of any one of claims 51-52, wherein the activatable anti-CD166 antibody-agent conjugate comprises a light chain and a heavy chain,
wherein the light chain comprises the prodomain and a VL, and wherein the light chain comprises the amino acid sequence of SEQ ID NO:127; and
wherein the heavy chain comprises the amino acid sequence of SEQ ID NO:126.

61. The ⁸⁹Zr-labeled activatable anti-CD166 antibody-agent conjugate of any one of claims 51-60, wherein the agent comprises DM4.

62. The ⁸⁹Zr-labeled activatable anti-CD166 antibody-agent conjugate of any of claims 51-61, wherein the ⁸⁹Zr is coupled to the activatable anti-CD166 antibody-agent conjugate via a chelation moiety having a structure corresponding to desferrioxamine.

63. The ⁸⁹Zr-labeled activatable anti-CD166 antibody-agent conjugate of any one of claims 51-62, wherein the activatable anti-CD166 antibody component of the conjugate comprises two identical light chains and two identical heavy chains.

64. A composition comprising the ⁸⁹Zr-labeled activatable anti-CD166 antibody-agent conjugate of any one of claims 51-63 and a pharmaceutically acceptable carrier.

65. A tracer dose comprising a pharmaceutically acceptable carrier and a quantity of the ⁸⁹Zr-labeled activatable anti-CD166 antibody-agent conjugate of any one of claims 51-63 corresponding to 37 MBq.


SEQUENCE LISTING (134 sequences)

SEQ ID NO: 1 - protein, 8 aa, Artificial Sequence
  Synthetic
LSGRSDNH

SEQ ID NO: 2 - protein, 8 aa, Artificial Sequence
  Synthetic
TGRGPSWV

SEQ ID NO: 3 - protein, 8 aa, Artificial Sequence
  Synthetic
PLTGRSGG

SEQ ID NO: 4 - protein, 8 aa, Artificial Sequence
  Synthetic
TARGPSFK

SEQ ID NO: 5 - protein, 12 aa, Artificial Sequence
  Synthetic
NTLSGRSENHSG

SEQ ID NO: 6 - protein, 12 aa, Artificial Sequence
  Synthetic
NTLSGRSGNHGS

SEQ ID NO: 7 - protein, 12 aa, Artificial Sequence
  Synthetic
TSTSGRSANPRG

SEQ ID NO: 8 - protein, 8 aa, Artificial Sequence
  Synthetic
TSGRSANP

SEQ ID NO: 9 - protein, 10 aa, Artificial Sequence
  Synthetic
VHMPLGFLGP

SEQ ID NO: 10 - protein, 8 aa, Artificial Sequence
  Synthetic
AVGLLAPP

SEQ ID NO: 11 - protein, 8 aa, Artificial Sequence
  Synthetic
AQNLLGMV

SEQ ID NO: 12 - protein, 8 aa, Artificial Sequence
  Synthetic
QNQALRMA

SEQ ID NO: 13 - protein, 8 aa, Artificial Sequence
  Synthetic
LAAPLGLL

SEQ ID NO: 14 - protein, 8 aa, Artificial Sequence
  Synthetic
STFPFGMF

SEQ ID NO: 15 - protein, 8 aa, Artificial Sequence
  Synthetic
ISSGLLSS

SEQ ID NO: 16 - protein, 8 aa, Artificial Sequence
  Synthetic
PAGLWLDP

SEQ ID NO: 17 - protein, 8 aa, Artificial Sequence
  Synthetic
VAGRSMRP

SEQ ID NO: 18 - protein, 8 aa, Artificial Sequence
  Synthetic
VVPEGRRS

SEQ ID NO: 19 - protein, 8 aa, Artificial Sequence
  Synthetic
ILPRSPAF

SEQ ID NO: 20 - protein, 8 aa, Artificial Sequence
  Synthetic
MVLGRSLL

SEQ ID NO: 21 - protein, 7 aa, Artificial Sequence
  Synthetic
QRAITFI

SEQ ID NO: 22 - protein, 8 aa, Artificial Sequence
  Synthetic
SPRSIMLA

SEQ ID NO: 23 - protein, 8 aa, Artificial Sequence
  Synthetic
SMLRSMPL

SEQ ID NO: 24 - protein, 13 aa, Artificial Sequence
  Synthetic
ISSGLLSGRSDNH

SEQ ID NO: 25 - protein, 18 aa, Artificial Sequence
  Synthetic
AVGLLAPPGGLSGRSDNH

SEQ ID NO: 26 - protein, 22 aa, Artificial Sequence
  Synthetic
ISSGLLSSGGSGGSLSGRSDNH

SEQ ID NO: 27 - protein, 8 aa, Artificial Sequence
  Synthetic
LSGRSGNH

SEQ ID NO: 28 - protein, 9 aa, Artificial Sequence
  Synthetic
SGRSANPRG

SEQ ID NO: 29 - protein, 8 aa, Artificial Sequence
  Synthetic
LSGRSDDH

SEQ ID NO: 30 - protein, 8 aa, Artificial Sequence
  Synthetic
LSGRSDIH

SEQ ID NO: 31 - protein, 8 aa, Artificial Sequence
  Synthetic
LSGRSDQH

SEQ ID NO: 32 - protein, 8 aa, Artificial Sequence
  Synthetic
LSGRSDTH

SEQ ID NO: 33 - protein, 8 aa, Artificial Sequence
  Synthetic
LSGRSDYH

SEQ ID NO: 34 - protein, 8 aa, Artificial Sequence
  Synthetic
LSGRSDNP

SEQ ID NO: 35 - protein, 8 aa, Artificial Sequence
  Synthetic
LSGRSANP

SEQ ID NO: 36 - protein, 8 aa, Artificial Sequence
  Synthetic
LSGRSANI

SEQ ID NO: 37 - protein, 8 aa, Artificial Sequence
  Synthetic
LSGRSDNI

SEQ ID NO: 38 - protein, 8 aa, Artificial Sequence
  Synthetic
MIAPVAYR

SEQ ID NO: 39 - protein, 8 aa, Artificial Sequence
  Synthetic
RPSPMWAY

SEQ ID NO: 40 - protein, 8 aa, Artificial Sequence
  Synthetic
WATPRPMR

SEQ ID NO: 41 - protein, 8 aa, Artificial Sequence
  Synthetic
FRLLDWQW

SEQ ID NO: 42 - protein, 5 aa, Artificial Sequence
  Synthetic
ISSGL

SEQ ID NO: 43 - protein, 7 aa, Artificial Sequence
  Synthetic
ISSGLLS

SEQ ID NO: 44 - protein, 6 aa, Artificial Sequence
  Synthetic
ISSGLL

SEQ ID NO: 45 - protein, 15 aa, Artificial Sequence
  Synthetic
ISSGLLSGRSANPRG

SEQ ID NO: 46 - protein, 18 aa, Artificial Sequence
  Synthetic
AVGLLAPPTSGRSANPRG

SEQ ID NO: 47 - protein, 17 aa, Artificial Sequence
  Synthetic
AVGLLAPPSGRSANPRG

SEQ ID NO: 48 - protein, 13 aa, Artificial Sequence
  Synthetic
ISSGLLSGRSDDH

SEQ ID NO: 49 - protein, 13 aa, Artificial Sequence
  Synthetic
ISSGLLSGRSDIH

SEQ ID NO: 50 - protein, 13 aa, Artificial Sequence
  Synthetic
ISSGLLSGRSDQH

SEQ ID NO: 51 - protein, 13 aa, Artificial Sequence
  Synthetic
ISSGLLSGRSDTH

SEQ ID NO: 52 - protein, 13 aa, Artificial Sequence
  Synthetic
ISSGLLSGRSDYH

SEQ ID NO: 53 - protein, 13 aa, Artificial Sequence
  Synthetic
ISSGLLSGRSDNP

SEQ ID NO: 54 - protein, 13 aa, Artificial Sequence
  Synthetic
ISSGLLSGRSANP

SEQ ID NO: 55 - protein, 13 aa, Artificial Sequence
  Synthetic
ISSGLLSGRSANI

SEQ ID NO: 56 - protein, 18 aa, Artificial Sequence
  Synthetic
AVGLLAPPGGLSGRSDDH

SEQ ID NO: 57 - protein, 18 aa, Artificial Sequence
  Synthetic
AVGLLAPPGGLSGRSDIH

SEQ ID NO: 58 - protein, 18 aa, Artificial Sequence
  Synthetic
AVGLLAPPGGLSGRSDQH

SEQ ID NO: 59 - protein, 18 aa, Artificial Sequence
  Synthetic
AVGLLAPPGGLSGRSDTH

SEQ ID NO: 60 - protein, 18 aa, Artificial Sequence
  Synthetic
AVGLLAPPGGLSGRSDYH

SEQ ID NO: 61 - protein, 18 aa, Artificial Sequence
  Synthetic
AVGLLAPPGGLSGRSDNP

SEQ ID NO: 62 - protein, 18 aa, Artificial Sequence
  Synthetic
AVGLLAPPGGLSGRSANP

SEQ ID NO: 63 - protein, 18 aa, Artificial Sequence
  Synthetic
AVGLLAPPGGLSGRSANI

SEQ ID NO: 64 - protein, 13 aa, Artificial Sequence
  Synthetic
ISSGLLSGRSDNI

SEQ ID NO: 65 - protein, 18 aa, Artificial Sequence
  Synthetic
AVGLLAPPGGLSGRSDNI

SEQ ID NO: 66 - protein, 19 aa, Artificial Sequence
  Synthetic
GLSGRSDNHGGAVGLLAPP

SEQ ID NO: 67 - protein, 21 aa, Artificial Sequence
  Synthetic
GLSGRSDNHGGVHMPLGFLGP

SEQ ID NO: 68 - protein, 5 aa, Artificial Sequence
  Synthetic
GSGGS

SEQ ID NO: 69 - protein, 4 aa, Artificial Sequence
  Synthetic
GGGS

SEQ ID NO: 70 - protein, 4 aa, Artificial Sequence
  Synthetic
GGSG

SEQ ID NO: 71 - protein, 5 aa, Artificial Sequence
  Synthetic
GGSGG

SEQ ID NO: 72 - protein, 5 aa, Artificial Sequence
  Synthetic
GSGSG

SEQ ID NO: 73 - protein, 5 aa, Artificial Sequence
  Synthetic
GSGGG

SEQ ID NO: 74 - protein, 5 aa, Artificial Sequence
  Synthetic
GGGSG

SEQ ID NO: 75 - protein, 5 aa, Artificial Sequence
  Synthetic
GSSSG

SEQ ID NO: 76 - protein, 13 aa, Artificial Sequence
  Synthetic
GSSGGSGGSGGSG

SEQ ID NO: 77 - protein, 11 aa, Artificial Sequence
  Synthetic
GSSGGSGGSGG

SEQ ID NO: 78 - protein, 12 aa, Artificial Sequence
  Synthetic
GSSGGSGGSGGS

SEQ ID NO: 79 - protein, 16 aa, Artificial Sequence
  Synthetic
GSSGGSGGSGGSGGGS

SEQ ID NO: 80 - protein, 10 aa, Artificial Sequence
  Synthetic
GSSGGSGGSG

SEQ ID NO: 81 - protein, 11 aa, Artificial Sequence
  Synthetic
GSSGGSGGSGS

SEQ ID NO: 82 - protein, 5 aa, Artificial Sequence
  Synthetic
GSSGT

SEQ ID NO: 83 - protein, 4 aa, Artificial Sequence
  Synthetic
GSSG

SEQ ID NO: 84 - protein, 15 aa, Artificial Sequence
  Synthetic
LCHPLVLSAWESCSS

SEQ ID NO: 85 - protein, 15 aa, Artificial Sequence
  Synthetic
LCHPAVLSAWESCSS

SEQ ID NO: 86 - protein, 15 aa, Artificial Sequence
  Synthetic
LCHPLVASAWESCSS

SEQ ID NO: 87 - protein, 15 aa, Artificial Sequence
  Synthetic
LEGWCLHPLCLWGAG

SEQ ID NO: 88 - protein, 15 aa, Artificial Sequence
  Synthetic
LCAPLVLSAWESCSS

SEQ ID NO: 89 - protein, 15 aa, Artificial Sequence
  Synthetic
LCHALVLSAWESCSS

SEQ ID NO: 90 - protein, 15 aa, Artificial Sequence
  Synthetic
LCHPLALSAWESCSS

SEQ ID NO: 91 - protein, 15 aa, Artificial Sequence
  Synthetic
LCHPLVLSAAESCSS

SEQ ID NO: 92 - protein, 15 aa, Artificial Sequence
  Synthetic
LCHPLVLSAWASCSS

SEQ ID NO: 93 - protein, 5 aa, Artificial Sequence
  Synthetic
HPLVL

SEQ ID NO: 94 - protein, 15 aa, Artificial Sequence
  Synthetic
LEGACLHPLCLWGAG

SEQ ID NO: 95 - protein, 15 aa, Artificial Sequence
  Synthetic
LEGWCAHPLCLWGAG

SEQ ID NO: 96 - protein, 15 aa, Artificial Sequence
  Synthetic
LEGWCLAPLCLWGAG

SEQ ID NO: 97 - protein, 14 aa, Artificial Sequence
  Synthetic
LEGWCLHACLWGAG

SEQ ID NO: 98 - protein, 15 aa, Artificial Sequence
  Synthetic
LEGWCLHPACLWGAG

SEQ ID NO: 99 - protein, 15 aa, Artificial Sequence
  Synthetic
LEGWCLHPLCAWGAG

SEQ ID NO: 100 - protein, 15 aa, Artificial Sequence
  Synthetic
LEGWCLHPLCLAGAG

SEQ ID NO: 101 - protein, 6 aa, Artificial Sequence
  Synthetic
CLHPLC

SEQ ID NO: 102 - protein, 6 aa, Artificial Sequence
  Synthetic
QGQSGQ

SEQ ID NO: 103 - protein, 7 aa, Artificial Sequence
  Synthetic
QGQSGQG

SEQ ID NO: 104 - protein, 5 aa, Artificial Sequence
  Synthetic
QGQSG

SEQ ID NO: 105 - protein, 4 aa, Artificial Sequence
  Synthetic
QGQS

SEQ ID NO: 106 - protein, 6 aa, Artificial Sequence
  Synthetic
GQSGQG

SEQ ID NO: 107 - protein, 5 aa, Artificial Sequence
  Synthetic
QSGQG

SEQ ID NO: 108 - protein, 4 aa, Artificial Sequence
  Synthetic
SGQG

SEQ ID NO: 109 - protein, 6 aa, Artificial Sequence
  Synthetic
GQSGQG

SEQ ID NO: 110 - protein, 5 aa, Artificial Sequence
  Synthetic
QSGQG

SEQ ID NO: 111 - protein, 4 aa, Artificial Sequence
  Synthetic
SGQG

SEQ ID NO: 112 - protein, 12 aa, Artificial Sequence
  Synthetic
GFSLSTYGMGVG

SEQ ID NO: 113 - protein, 9 aa, Artificial Sequence
  Synthetic
NIWWSEDKH

SEQ ID NO: 114 - protein, 11 aa, Artificial Sequence
  Synthetic
IDYGNDYAFTY

SEQ ID NO: 115 - protein, 16 aa, Artificial Sequence
  Synthetic
RSSKSLLHSNGITYLY

SEQ ID NO: 116 - protein, 7 aa, Artificial Sequence
  Synthetic
QMSNLAS

SEQ ID NO: 117 - protein, 9 aa, Artificial Sequence
  Synthetic
AQNLELPYT

SEQ ID NO: 118 - protein, 121 aa, Artificial Sequence
  Synthetic
QITLKESGPTLVKPTQTLTLTCTFSGFSLSTYGMGVGWIRQPPGKALEWLANIWWSEDKHYSPSLKSRLTITKDTSKNQVVLTMTNMDPVDTATYYCVQIDYGNDYAFTYWGQGTLVTVSS

SEQ ID NO: 119 - protein, 121 aa, Artificial Sequence
  Synthetic
QITLKESGPTLVKPTQTLTLTCTFSGFSLSTYGMGVGWIRQPPGKALEWLANIWWSEDKHYSPSLKSRLTITKDTSKNQVVLTITNVDPVDTATYYCVQIDYGNDYAFTYWGQGTLVTVSS

SEQ ID NO: 120 - protein, 112 aa, Artificial Sequence
  Synthetic
DIVMTQSPLSLPVTPGEPASISCRSSKSLLHSNGITYLYWYLQKPGQSPQLLIYQMSNLASGVPDRFSGSGSGTDFTLKISRVEAEDVGVYYCAQNLELPYTFGQGTKLEIK

SEQ ID NO: 121 - protein, 112 aa, Artificial Sequence
  Synthetic
DIVMTQSPLSLPVTPGEPASISCRSSKSLLHSNGITYLYWYLQKPGQSPQLLIYQMSNLASGVPDRFSSSGSGTDFTLKISRVEAEDVGVYYCAQNLELPYTFGQGTKLEIK

SEQ ID NO: 122 - protein, 112 aa, Artificial Sequence
  Synthetic
DIVMTQSPLSLPVTPGEPASISCRSSQSLLHSNGITYLYWYLQKPGQSPQLLIYQMSNRASGVPDRFSGSGSGTDFTLKISRVEAEDVGVYYCAQNLELPYTFGQGTKLEIK

SEQ ID NO: 123 - protein, 112 aa, Artificial Sequence
  Synthetic
DIVMTQSPLSLPVTPGEPASISCRSSQSLLHSNGITYLYWYLQKPGQSPQLLIYQMSNRASGVPDRFSSSGSGTDFTLKISRVEAEDVGVYYCAQNLELPYTFGQGTKLEIK

SEQ ID NO: 124 - protein, 16 aa, Artificial Sequence
  Synthetic
RSSQSLLHSNGITYLY

SEQ ID NO: 125 - protein, 7 aa, Artificial Sequence
  Synthetic
QMSNRAS

SEQ ID NO: 126 - protein, 450 aa, Artificial Sequence
  Synthetic
QITLKESGPTLVKPTQTLTLTCTFSGFSLSTYGMGVGWIRQPPGKALEWLANIWWSEDKHYSPSLKSRLTITKDTSKNQVVLTITNVDPVDTATYYCVQIDYGNDYAFTYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 127 - protein, 270 aa, Artificial Sequence
  Synthetic
QGQSGQGLCHPAVLSAWESCSSGGGSSGGSAVGLLAPPGGLSGRSDNHGGSDIVMTQSPLSLPVTPGEPASISCRSSKSLLHSNGITYLYWYLQKPGQSPQLLIYQMSNLASGVPDRFSGSGSGTDFTLKISRVEAEDVGVYYCAQNLELPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 128 - protein, 13 aa, Artificial Sequence
  Synthetic
GGGSSGGSGGSGG

SEQ ID NO: 129 - protein, 6 aa, Artificial Sequence
  Synthetic
QGQSGS

SEQ ID NO: 130 - protein, 5 aa, Artificial Sequence
  Synthetic
GQSGS

SEQ ID NO: 131 - protein, 4 aa, Artificial Sequence
  Synthetic
QSGS

SEQ ID NO: 132 - protein, 5 aa, Artificial Sequence
  Synthetic
GQSGQ

SEQ ID NO: 133 - protein, 4 aa, Artificial Sequence
  Synthetic
QSGQ

SEQ ID NO: 134 - protein, 583 aa, Artificial Sequence
  Synthetic
MESKGASSCRLLFCLLISATVFRPGLGWYTVNSAYGDTIIIPCRLDVPQNLMFGKWKYEKPDGSPVFIAFRSSTKKSVQYDDVPEYKDRLNLSENYTLSISNARISDEKRFVCMLVTEDNVFEAPTIVKVFKQPSKPEIVSKALFLETEQLKKLGDCISEDSYPDGNITWYRNGKVLHPLEGAVVIIFKKEMDPVTQLYTMTSTLEYKTTKADIQMPFTCSVTYYGPSGQKTIHSEQAVFDIYYPTEQVTIQVLPPKNAIKEGDNITLKCLGNGNPPPEEFLFYLPGQPEGIRSSNTYTLMDVRRNATGDYKCSLIDKKSMIASTAITVHYLDLSLNPSGEVTRQIGDALPVSCTISASRNATVVWMKDNIRLRSSPSFSSLHYQDAGNYVCETALQEVEGLKKRESLTLIVEGKPQIKMTKKTDPSGLSKTIICHVEGFPKPAIQWTITGSGSVINQTEESPYINGRYYSKIIISPEENVTLTCTAENQLERTVNSLNVSAISIPEHDEADEISDENREKVNDQAKLIVGIVVGLLLAALVAGVVYWLYMKKSKTASKHVNKDLGNMEENKKLEENNHKTEA